(19) United States
(12) Patent Application Publication
(10) Pub. No.: US 2026/0295365 A1
(43) Pub. Date: Oct. 1, 2026

(54) HEALTH AND FITNESS TRACKING USING MULTI-SENSOR DATA FROM A HEAD-WEARABLE DEVICE, AND SYSTEMS AND METHODS OF USE THEREOF

(71) Applicant: Meta Platforms Technologies, LLC, Menlo Park, CA (US)

(72) Inventors: Venkataraman Chandrasekaran, Chicago, IL (US); Sneha Kadetotad, Sunnyvale, CA (US); Vamshi Kandala, Redmond, WA (US); Dmitri Model, Nashville, TN (US); Jennifer Monti, Yarmouth, ME (US); Bharath Kumar Narahari, Fremont, CA (US); Akhil Amar Padmanabha, San Jose, CA (US); Pavana Sirimamilla, Sammamish, WA (US); Mantas Zurauskas, Redmond, WA (US)

(21) Appl. No.: 19/633,946

(22) Filed: Mar. 30, 2026

Related U.S. Application Data
(60) Provisional application No. 63/780,552, filed on Mar. 31, 2025.

(51) Int. Cl. A63B 71/06; A63B 24/00

(52) U.S. Cl. CPC A63B 71/0622; A63B 24/0006; A63B 2024/0071; A63B 2071/0666

(57) ABSTRACT

A method of a user wearing a head-wearable device with a plurality of sensors while exercising is described. The method includes, receiving, from a sensor located on a frame of the head-wearable device, the sensor configured to collect data corresponding to an attribute of a head of the user, data, determining a metric corresponding to an attribute of a body of the user based on the data corresponding to the attribute of the head of the user, determining whether a difference between the metric and an expected metric corresponding to the attribute of the body of the user satisfies a predetermined threshold, and responsive to determining that the difference satisfies the predetermined threshold, causing presentation of an indication pertaining to the attribute of the body of the user.

### RELATED APPLICATIONS

This application claims priority to U.S. Provisional Patent App. No. 63/780,552, filed Mar. 31, 2025, entitled “Fitness Tracking Using Multi-Sensor Data from Head Mounted Device,” which is incorporated herein by reference.

### TECHNICAL FIELD

This relates generally to wearable devices, more specifically, tracking and monitoring health and fitness data from sensors on a head-wearable device.

### BACKGROUND

Health and fitness data for wearable devices is typically limited to post-activity feedback after all data has been captured and analyzed as a whole. Additionally, health and fitness tracking may introduce distractions with real-time notifications (e.g., looking down at a display and/or providing feedback as a reaction to user input, rather than proactively), which may decrease the usefulness of the notifications.

As such, there is a need to address one or more of the above-identified challenges. A brief summary of solutions to the issues noted above are described below.

### SUMMARY

The systems and methods disclosed herein provide wearable devices that can quickly provide an output based on user health data, the data provided in various modalities. The systems and methods disclosed herein allow users to focus on their workout or activity, rather than be distracted by inputting commands at their wearable device to provide a specific output. One example of an extended-reality (XR) device including a frame configured to be worn on a head of the user and a sensor located on the frame, the sensor configured to collect data corresponding to an attribute of the head of the user, is described herein. This example XR device includes a processor programmed to perform operations. The operations include, receive the data from the sensor, determine a metric corresponding to an attribute of a body of the user based on the data corresponding to the attribute of the head of the user, determine whether a difference between the metric and an expected metric corresponding to the attribute of the body of the user satisfies a predetermined threshold, and responsive to determining that the difference satisfies the predetermined threshold, cause presentation of an indication pertaining to the attribute of the body of the user.

Instructions that cause performance of the methods and operations described herein can be stored on a non-transitory computer-readable storage medium. The non-transitory computer-readable storage medium can be included on a single electronic device or spread across multiple electronic devices of a system (computing system). A non-exhaustive list of electronic devices that can either alone or in combination (e.g., a system) perform the method and operations described herein include an extended-reality (XR) headset/glasses (e.g., a mixed-reality (MR) headset or a pair of augmented-reality (AR) glasses as two examples), a wrist-wearable device, an intermediary processing device, a smart textile-based garment, etc. For instance, the instructions can be stored on a pair of AR glasses or can be stored on a combination of a pair of AR glasses and an associated input device (e.g., a wrist-wearable device) such that instructions for causing detection of input operations can be performed at the input device and instructions for causing changes to a displayed user interface in response to those input operations can be performed at the pair of AR glasses. The devices and systems described herein can be configured to be used in conjunction with methods and operations for providing an XR experience. The methods and operations for providing an XR experience can be stored on a non-transitory computer-readable storage medium.

The devices and/or systems described herein can be configured to include instructions that cause the performance of methods and operations associated with the presentation and/or interaction with an extended-reality (XR) headset. These methods and operations can be stored on a non-transitory computer-readable storage medium of a device or a system. It is also noted that the devices and systems described herein can be part of a larger, overarching system that includes multiple devices. A non-exhaustive of list of electronic devices that can, either alone or in combination (e.g., a system), include instructions that cause the performance of methods and operations associated with the presentation and/or interaction with an XR experience include an extended-reality headset (e.g., a mixed-reality (MR) headset or a pair of augmented-reality (AR) glasses as two examples), a wrist-wearable device, an intermediary processing device, a smart textile-based garment, etc. For example, when an XR headset is described, it is understood that the XR headset can be in communication with one or more other devices (e.g., a wrist-wearable device, a server, intermediary processing device) which together can include instructions for performing methods and operations associated with the presentation and/or interaction with an extended-reality system (i.e., the XR headset would be part of a system that includes one or more additional devices). Multiple combinations with different related devices are envisioned, but not recited for brevity.

Fitness in virtual reality (VR) is one of the most popular use cases of MR headsets. In a VR training environment, effectively optimizing fitness activities and extracting meaningful biometrics presents a complex challenge. The integration of diverse sensor data, including heart rate via imaging photoplethysmography (iPPG), pupil dilation, microphone recordings of breathing patterns, body movement tracking, and activity-type recognition, requires a sophisticated approach to ensure accuracy and usability. The goal is to enhance user engagement, ensure safety, and provide personalized training experiences that adapt dynamically to the fitness levels and goals of the user.

To optimize fitness activity in VR training, the system may utilize the collected data to create personalized training programs that adjust in real time based on the user's biometric feedback. For instance, if heart rate data indicates suboptimal exertion levels, the VR system may automatically intensify the workout. Additionally, the system may provide real-time feedback on performance and safety, offer engaging and motivating VR scenarios to enhance user experience, and continuously monitor for any signs of health risks, adjusting the training regimen accordingly.

In a VR training environment, optimizing fitness activity and extracting meaningful biometrics involves a comprehensive approach that integrates data from various sensors, including heart rate via iPPG, microphone recordings of breathing patterns, body movement tracking, and activity-type recognition. Biometrics may be extracted, and VR fitness may be enhanced in various ways. Enhancing fitness activity in VR training may involve the use of personalized training programs that utilize the collected data to create personalized training sessions that adapt to the user's fitness level and goals. For example, if the heart rate data indicates that the user is not reaching the desired intensity, the VR system may increase the difficulty of the exercise dynamically. Additionally, real-time feedback may be provided to the user based on the biometric data. For instance, if the breathing pattern becomes irregular or too strained, the system may suggest that the user decrease intensity or take a break. Also, VR environments may be used to simulate engaging and motivating scenarios that encourage longer or more frequent workouts. For example, engaging and motivating scenarios may include changing virtual landscapes, gamifying fitness challenges, or providing virtual workout companions. Further, safety monitoring may be performed by, for example, continuously monitoring biometric data to ensure the user's safety and alerting the user if any biometric signals indicate potential health risks, such as extremely high heart rate or signs of respiratory distress. Finally, real-time feedback may be provided, such as calories expenditure and/or time duration spend training in each “heart rate zone.”

Various types of meaningful biometrics may be extracted. For example, heart rate and variability (HRV) may be extracted to monitor cardiovascular strain and recovery. HRV may provide insights into the user's stress level and cardiovascular fitness. Additionally, breathing rate and depth may be extracted by analyzing microphone recordings to assess breathing patterns. This may help in determining respiratory efficiency and the aerobic intensity of the workout. Also, motion analysis may be performed using data from body tracking cameras to analyze movement efficiency, form, and technique. This may help in correcting posture, reducing the risk of injury, and improving the effectiveness of the workout. Further, energy expenditure may be estimated as a number of calories burned based on the intensity and type of activity combined with heart rate data. Further, stress and recovery levels may be determined using changes in HRV and breathing patterns to estimate stress levels during exercise and recovery times. Finally, progress tracking may be performed by analyzing trends in all collected biometrics to monitor improvements in fitness levels over time, such as increases in workout intensity, improvements in breathing efficiency, and heart rate recovery.

The features and advantages described in the specification are not necessarily all inclusive and, in particular, certain additional features and advantages will be apparent to one of ordinary skill in the art in view of the drawings, specification, and claims. Moreover, it should be noted that the language used in the specification has been principally selected for readability and instructional purposes.

Having summarized the above example aspects, a brief description of the drawings will now be presented.

### BRIEF DESCRIPTION OF THE DRAWINGS

For a better understanding of the various described embodiments, reference should be made to the Detailed Description below, in conjunction with the following drawings in which like reference numerals refer to corresponding parts throughout the figures.

FIG. 1 illustrates a user wearing a head-wearable device with a plurality of sensors, in accordance with some embodiments.

FIGS. 2A-2D illustrate a user wearing a head-wearable device with a plurality of sensors while exercising, in accordance with some embodiments.

FIG. 3 shows an example method flow chart for a user wearing a head-wearable device with a plurality of sensors while exercising, in accordance with some embodiments.

FIG. 4 illustrates data collected from a head-wearable device with a plurality of sensors while a user wearing the head-wearable device exercises, in accordance with some embodiments.

FIG. 5 shows an example method flow chart for a user wearing a head-wearable device with a plurality of sensors while exercising, in accordance with some embodiments.

FIG. 6 shows an example method flow chart for a user wearing a head-wearable device with a plurality of sensors while exercising, in accordance with some embodiments.

FIGS. 7A and 7B illustrate pupil dilation correlation with VO₂ MAX, in accordance with some embodiments.

FIGS. 8A and 8B illustrate a user wearing a head-wearable device with a plurality of sensors, in accordance with some embodiments.

FIG. 9 shows an example method flow chart for a user wearing a head-wearable device with a plurality of sensors while exercising, in accordance with some embodiments.

FIGS. 10A, 10B, 10C-1, and 10C-2 illustrate example MR and AR systems, in accordance with some embodiments.

In accordance with common practice, the various features illustrated in the drawings may not be drawn to scale. Accordingly, the dimensions of the various features may be arbitrarily expanded or reduced for clarity. In addition, some of the drawings may not depict all of the components of a given system, method, or device. Finally, like reference numerals may be used to denote like features throughout the specification and figures.

### DETAILED DESCRIPTION

Numerous details are described herein to provide a thorough understanding of the example embodiments illustrated in the accompanying drawings. However, some embodiments may be practiced without many of the specific details, and the scope of the claims is only limited by those features and aspects specifically recited in the claims. Furthermore, well-known processes, components, and materials have not necessarily been described in exhaustive detail so as to avoid obscuring pertinent aspects of the embodiments described herein.

### Overview

Embodiments of this disclosure can include or be implemented in conjunction with various types of extended-realities (XRs) such as mixed-reality (MR) and augmented-reality (AR) systems. MRs and ARs, as described herein, are any superimposed functionality and/or sensory-detectable presentation provided by MR and AR systems within a user's physical surroundings. Such MRs can include and/or represent virtual realities (VRs) and VRs in which at least some aspects of the surrounding environment are reconstructed within the virtual environment (e.g., displaying virtual reconstructions of physical objects in a physical environment to avoid the user colliding with the physical objects in a surrounding physical environment). In the case of MRs, the surrounding environment that is presented through a display is captured via one or more sensors configured to capture the surrounding environment (e.g., a camera sensor, time-of-flight (ToF) sensor). While a wearer of an MR headset can see the surrounding environment in full detail, they are seeing a reconstruction of the environment reproduced using data from the one or more sensors (i.e., the physical objects are not directly viewed by the user). An MR headset can also forgo displaying reconstructions of objects in the physical environment, thereby providing a user with an entirely VR experience. An AR system, on the other hand, provides an experience in which information is provided, e.g., through the use of a waveguide, in conjunction with the direct viewing of at least some of the surrounding environment through a transparent or semi-transparent waveguide(s) and/or lens(es) of the AR glasses. Throughout this application, the term “extended reality (XR)” is used as a catchall term to cover both ARs and MRs. In addition, this application also uses, at times, a head-wearable device or headset device as a catchall term that covers XR headsets such as AR glasses and MR headsets.

As alluded to above, an MR environment, as described herein, can include, but is not limited to, non-immersive, semi-immersive, and fully immersive VR environments. As also alluded to above, AR environments can include marker-based AR environments, markerless AR environments, location-based AR environments, and projection-based AR environments. The above descriptions are not exhaustive and any other environment that allows for intentional environmental lighting to pass through to the user would fall within the scope of an AR, and any other environment that does not allow for intentional environmental lighting to pass through to the user would fall within the scope of an MR.

The AR and MR content can include video, audio, haptic events, sensory events, or some combination thereof, any of which can be presented in a single channel or in multiple channels (such as stereo video that produces a three-dimensional effect to a viewer). Additionally, AR and MR can also be associated with applications, products, accessories, services, or some combination thereof, which are used, for example, to create content in an AR or MR environment and/or are otherwise used in (e.g., to perform activities in) AR and MR environments.

Interacting with these AR and MR environments described herein can occur using multiple different modalities and the resulting outputs can also occur across multiple different modalities. In one example AR or MR system, a user can perform a swiping in-air hand gesture to cause a song to be skipped by a song-providing application programming interface (API) providing playback at, for example, a home speaker.

A hand gesture, as described herein, can include an in-air gesture, a surface-contact gesture, and or other gestures that can be detected and determined based on movements of a single hand (e.g., a one-handed gesture performed with a user's hand that is detected by one or more sensors of a wearable device (e.g., electromyography (EMG) and/or inertial measurement units (IMUs) of a wrist-wearable device, and/or one or more sensors included in a smart textile wearable device) and/or detected via image data captured by an imaging device of a wearable device (e.g., a camera of a head-wearable device, an external tracking camera setup in the surrounding environment)). “In-air” generally includes gestures in which the user's hand does not contact a surface, object, or portion of an electronic device (e.g., a head-wearable device or other communicatively coupled device, such as the wrist-wearable device), in other words the gesture is performed in open air in 3D space and without contacting a surface, an object, or an electronic device. Surface-contact gestures (contacts at a surface, object, body part of the user, or electronic device) more generally are also contemplated in which a contact (or an intention to contact) is detected at a surface (e.g., a single-or double-finger tap on a table, on a user's hand or another finger, on the user's leg, a couch, a steering wheel). The different hand gestures disclosed herein can be detected using image data and/or sensor data (e.g., neuromuscular signals sensed by one or more biopotential sensors (e.g., EMG sensors) or other types of data from other sensors, such as proximity sensors, ToF sensors, sensors of an IMU, capacitive sensors, strain sensors) detected by a wearable device worn by the user and/or other electronic devices in the user's possession (e.g., smartphones, laptops, imaging devices, intermediary devices, and/or other devices described herein).

The input modalities as alluded to above can be varied and are dependent on a user's experience. For example, in an interaction in which a wrist-wearable device is used, a user can provide inputs using in-air or surface-contact gestures that are detected using neuromuscular signal sensors of the wrist-wearable device. In the event that a wrist-wearable device is not used, alternative and entirely interchangeable input modalities can be used instead, such as camera(s) located on the headset/glasses or elsewhere to detect in-air or surface-contact gestures or inputs at an intermediary processing device (e.g., through physical input components (e.g., buttons and trackpads)). These different input modalities can be interchanged based on both desired user experiences, portability, and/or a feature set of the product (e.g., a low-cost product may not include hand-tracking cameras).

While the inputs are varied, the resulting outputs stemming from the inputs are also varied. For example, an in-air gesture input detected by a camera of a head-wearable device can cause an output to occur at a head-wearable device or control another electronic device different from the head-wearable device. In another example, an input detected using data from a neuromuscular signal sensor can also cause an output to occur at a head-wearable device or control another electronic device different from the head-wearable device. While only a couple examples are described above, one skilled in the art would understand that different input modalities are interchangeable along with different output modalities in response to the inputs.

Specific operations described above may occur as a result of specific hardware. The devices described are not limiting and features on these devices can be removed or additional features can be added to these devices. The different devices can include one or more analogous hardware components. For brevity, analogous devices and components are described herein. Any differences in the devices and components are described below in their respective sections.

As described herein, a processor (e.g., a central processing unit (CPU) or microcontroller unit (MCU)), is an electronic component that is responsible for executing instructions and controlling the operation of an electronic device (e.g., a wrist-wearable device, a head-wearable device, a handheld intermediary processing device (HIPD), a smart textile-based garment, or other computer system). There are various types of processors that may be used interchangeably or specifically required by embodiments described herein. For example, a processor may be (i) a general processor designed to perform a wide range of tasks, such as running software applications, managing operating systems, and performing arithmetic and logical operations; (ii) a microcontroller designed for specific tasks such as controlling electronic devices, sensors, and motors; (iii) a graphics processing unit (GPU) designed to accelerate the creation and rendering of images, videos, and animations (e.g., VR animations, such as three-dimensional modeling); (iv) a field-programmable gate array (FPGA) that can be programmed and reconfigured after manufacturing and/or customized to perform specific tasks, such as signal processing, cryptography, and machine learning; or (v) a digital signal processor (DSP) designed to perform mathematical operations on signals such as audio, video, and radio waves. One of skill in the art will understand that one or more processors of one or more electronic devices may be used in various embodiments described herein.

As described herein, controllers are electronic components that manage and coordinate the operation of other components within an electronic device (e.g., controlling inputs, processing data, and/or generating outputs). Examples of controllers can include (i) microcontrollers, including small, low-power controllers that are commonly used in embedded systems and Internet of Things (IoT) devices; (ii) programmable logic controllers (PLCs) that may be configured to be used in industrial automation systems to control and monitor manufacturing processes; (iii) system-on-a-chip (SoC) controllers that integrate multiple components such as processors, memory, I/O interfaces, and other peripherals into a single chip; and/or (iv) DSPs. As described herein, a graphics module is a component or software module that is designed to handle graphical operations and/or processes and can include a hardware module and/or a software module.

As described herein, memory refers to electronic components in a computer or electronic device that store data and instructions for the processor to access and manipulate. The devices described herein can include volatile and non-volatile memory. Examples of memory can include (i) random access memory (RAM), such as DRAM, SRAM, DDR RAM or other random access solid state memory devices, configured to store data and instructions temporarily; (ii) read-only memory (ROM) configured to store data and instructions permanently (e.g., one or more portions of system firmware and/or boot loaders); (iii) flash memory, magnetic disk storage devices, optical disk storage devices, other non-volatile solid state storage devices, which can be configured to store data in electronic devices (e.g., universal serial bus (USB) drives, memory cards, and/or solid-state drives (SSDs)); and (iv) cache memory configured to temporarily store frequently accessed data and instructions. Memory, as described herein, can include structured data (e.g., SQL databases, MongoDB databases, GraphQL data, or JSON data). Other examples of memory can include (i) profile data, including user account data, user settings, and/or other user data stored by the user; (ii) sensor data detected and/or otherwise obtained by one or more sensors; (iii) media content data including stored image data, audio data, documents, and the like; (iv) application data, which can include data collected and/or otherwise obtained and stored during use of an application; and/or (v) any other types of data described herein.

As described herein, a power system of an electronic device is configured to convert incoming electrical power into a form that can be used to operate the device. A power system can include various components, including (i) a power source, which can be an alternating current (AC) adapter or a direct current (DC) adapter power supply; (ii) a charger input that can be configured to use a wired and/or wireless connection (which may be part of a peripheral interface, such as a USB, micro-USB interface, near-field magnetic coupling, magnetic inductive and magnetic resonance charging, and/or radio frequency (RF) charging); (iii) a power-management integrated circuit, configured to distribute power to various components of the device and ensure that the device operates within safe limits (e.g., regulating voltage, controlling current flow, and/or managing heat dissipation); and/or (iv) a battery configured to store power to provide usable power to components of one or more electronic devices.

As described herein, peripheral interfaces are electronic components (e.g., of electronic devices) that allow electronic devices to communicate with other devices or peripherals and can provide a means for input and output of data and signals. Examples of peripheral interfaces can include (i) USB and/or micro-USB interfaces configured for connecting devices to an electronic device; (ii) Bluetooth interfaces configured to allow devices to communicate with each other, including Bluetooth low energy (BLE); (iii) near-field communication (NFC) interfaces configured to be short-range wireless interfaces for operations such as access control; (iv) pogo pins, which may be small, spring-loaded pins configured to provide a charging interface; (v) wireless charging interfaces; (vi) global-positioning system (GPS) interfaces; (vii) Wi-Fi interfaces for providing a connection between a device and a wireless network; and (viii) sensor interfaces.

As described herein, sensors are electronic components (e.g., in and/or otherwise in electronic communication with electronic devices, such as wearable devices) configured to detect physical and environmental changes and generate electrical signals. Examples of sensors can include (i) imaging sensors for collecting imaging data (e.g., including one or more cameras disposed on a respective electronic device, such as a simultaneous localization and mapping (SLAM) camera); (ii) biopotential-signal sensors (used interchangeably with neuromuscular-signal sensors); (iii) IMUs for detecting, for example, angular rate, force, magnetic field, and/or changes in acceleration; (iv) heart rate sensors for measuring a user's heart rate; (v) peripheral oxygen saturation (SpO₂) sensors for measuring blood oxygen saturation and/or other biometric data of a user; (vi) capacitive sensors for detecting changes in potential at a portion of a user's body (e.g., a sensor-skin interface) and/or the proximity of other devices or objects; (vii) sensors for detecting some inputs (e.g., capacitive and force sensors); and (viii) light sensors (e.g., ToF sensors, infrared light sensors, or visible light sensors), and/or sensors for sensing data from the user or the user's environment. As described herein biopotential-signal-sensing components are devices used to measure electrical activity within the body (e.g., biopotential-signal sensors). Some types of biopotential-signal sensors include (i) electroencephalography (EEG) sensors configured to measure electrical activity in the brain to diagnose neurological disorders; (ii) electrocardiography (ECG or EKG) sensors configured to measure electrical activity of the heart to diagnose heart problems; (iii) EMG sensors configured to measure the electrical activity of muscles and diagnose neuromuscular disorders; (iv) electrooculography (EOG) sensors configured to measure the electrical activity of eye muscles to detect eye movement and diagnose eye disorders.

As described herein, an application stored in memory of an electronic device (e.g., software) includes instructions stored in the memory. Examples of such applications include (i) games; (ii) word processors; (iii) messaging applications; (iv) media-streaming applications; (v) financial applications; (vi) calendars; (vii) clocks; (viii) web browsers; (ix) social media applications; (x) camera applications; (xi) web-based applications; (xii) health applications; (xiii) AR and MR applications; and/or (xiv) any other applications that can be stored in memory. The applications can operate in conjunction with data and/or one or more components of a device or communicatively coupled devices to perform one or more operations and/or functions.

As described herein, communication interface modules can include hardware and/or software capable of data communications using any of a variety of custom or standard wireless protocols (e.g., IEEE 802.15.4, Wi-Fi, ZigBee, 6LoWPAN, Thread, Z-Wave, Bluetooth Smart, ISA100.11a, WirelessHART, or MiWi), custom or standard wired protocols (e.g., Ethernet or HomePlug), and/or any other suitable communication protocol, including communication protocols not yet developed as of the filing date of this document. A communication interface is a mechanism that enables different systems or devices to exchange information and data with each other, including hardware, software, or a combination of both hardware and software. For example, a communication interface can refer to a physical connector and/or port on a device that enables communication with other devices (e.g., USB, Ethernet, HDMI, or Bluetooth). A communication interface can refer to a software layer that enables different software programs to communicate with each other (e.g., APIs and protocols such as HTTP and TCP/IP).

As described herein, a graphics module is a component or software module that is designed to handle graphical operations and/or processes and can include a hardware module and/or a software module.

As described herein, non-transitory computer-readable storage media are physical devices or storage medium that can be used to store electronic data in a non-transitory form (e.g., such that the data is stored permanently until it is intentionally deleted and/or modified).

### Using Data From Multiple Sensors on a Head-wearable Device Obtained During a Workout

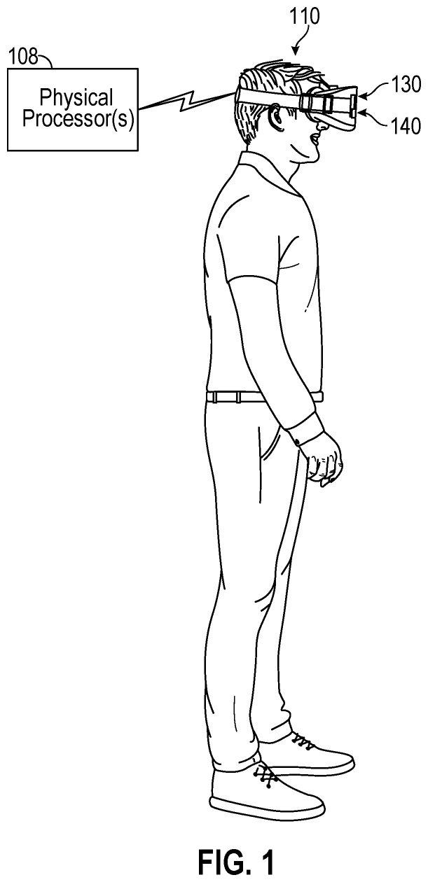

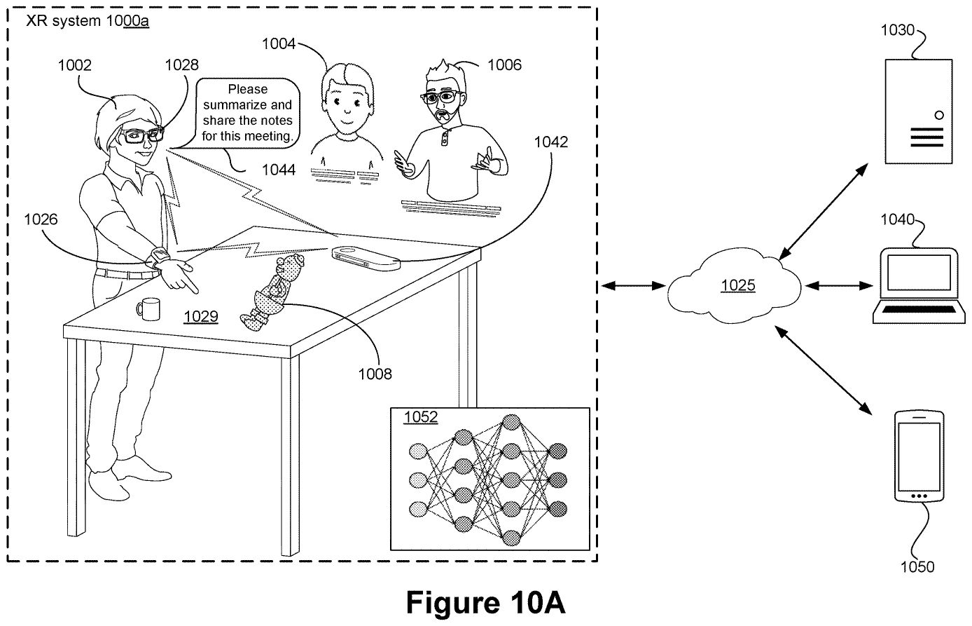

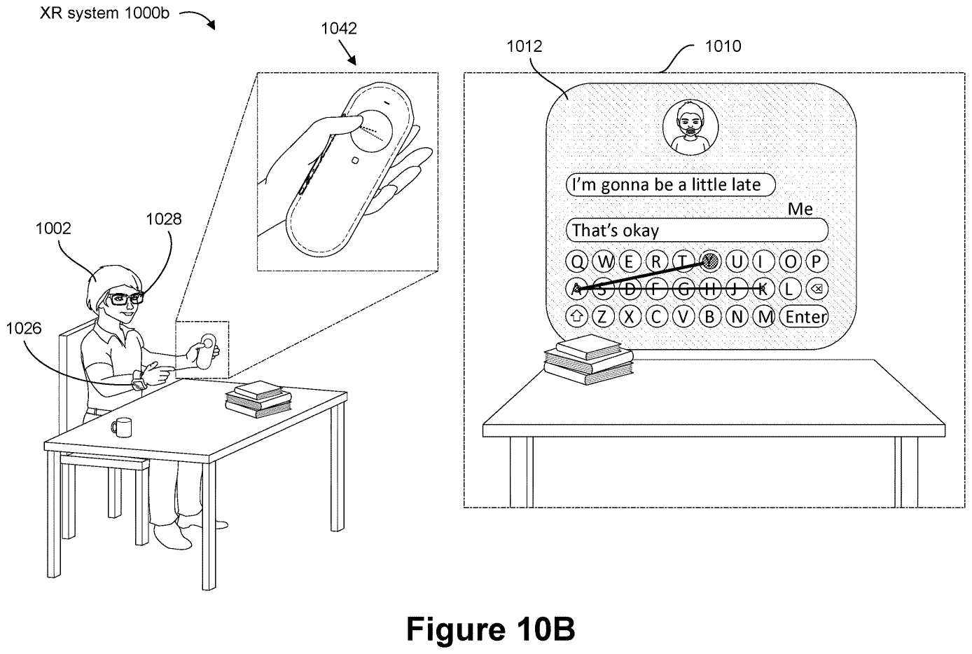

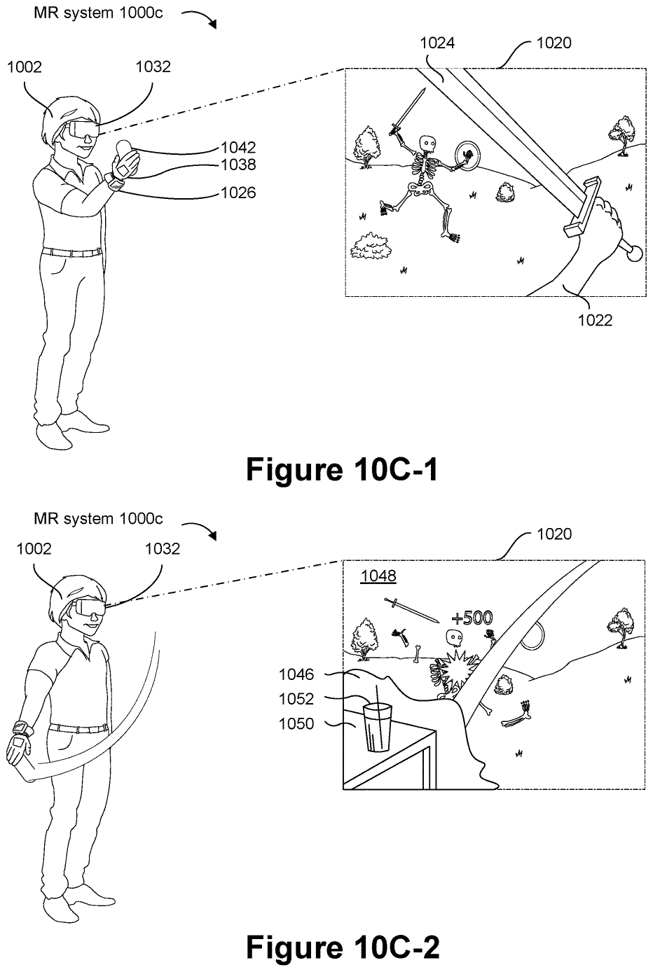

FIGS. 1-9 illustrates a user 110 performing an activity (e.g., a workout, or a physical movement of a portion or all of the body of the user 110) while wearing a head-wearable device 130 with a plurality of sensors 140 (e.g., biopotential sensors (e.g., EMG sensors), proximity sensors, ToF sensors, sensors of an IMU, capacitive sensors, strain sensors, heart rate sensors, altitude sensors, altimeters, gait sensors, eye-tracking sensors, positional sensors, microphones, cameras, GPS sensors, temperature sensors, inertial measurement units (IMUs), wear detectors, peripheral oxygen saturation (SpO2) sensors, etc.), in accordance with some embodiments. As described below in reference to FIG. 10A, the head-wearable device 130 can include one or more displays, an imaging device (e.g., a camera), a microphone, a speaker, input surfaces (e.g., touch input surfaces, mechanical inputs, etc.), and the plurality of sensors 140. In some embodiments, the head-wearable device is one or more of a pair of smart glasses, a smart head-wearable device, an AR headset, a VR headset, an MR headset, an XR headset, and/or another electronic device worn on a head and/or face of the user 110. In some embodiments, the head-wearable device 130 is communicatively coupled via a network (e.g., a cellular network, a near field connection, a Wi-Fi connection, a personal area network, etc.) to an additional wearable device (e.g., a smart watch, a smart textile-based garment such as wearable bands, shirts, etc.). Additionally, the head-wearable device 130 is further communicatively coupled to another electronic device, such as a server device, a handheld intermediary processing device (HIPD) (e.g., the HIPD 1042 described in reference to FIGS. 10A-10B), a computer (e.g., the computer 1040, described in reference to FIG. 10A), mobile devices (e.g., mobile devices 1050, described in reference to FIG. 10A), and/or other electronic devices described below in reference to FIGS. 10A-10C. The wearable devices and the electronic devices can be communicatively coupled via the network.

FIG. 1 illustrates the user 110 wearing the head-wearable device 130 with the plurality of sensors 140 located on a frame of the head-wearable device 130, in accordance with some embodiments. In some embodiments, the plurality of sensors 140 includes one or more of heart rate sensors, altitude sensors (e.g., altimeters), gait sensors, eye-tracking sensors, positional sensors, microphones, cameras, GPS sensors, temperature sensors, IMUs, wear detectors, SpO2 sensors, proximity sensors, ToF sensors, capacitive sensors, strain sensors, and EMG sensors. In some embodiments the heart rate sensors detect a heart rate of the user 110. In some embodiments, the altitude sensor detects a change in altitude of the user 110 relative to a starting position. In some embodiments, the altitude sensors detect a vertical oscillation of the user 110 relative to a starting position. In some embodiments, the gait sensors capture a gait of the user 110 as they are running, jogging, walking, cycling, or performing other activities. In some embodiments, the eye-tracking sensors capture a pupil dilation of the user 110. In some embodiments, the pupil dilation of the user 110 is used to estimate a cardiovascular strain of the user 110, and a stress level of the user 110 is calculated based on the estimated cardiovascular strain. In some embodiments, the positional sensors determine a body position of the user 110 over a period of time to calculate movement efficiency of the user 110 during the activity. In some embodiments, the positional sensors determine a head position of the user 110 over the period of time. In some embodiments, the microphones capture breathing data from the user 110 and analyze the breathing patterns to monitor respiratory efficiency during the activity. In some embodiments, the cameras capture image data to analyze safety conditions surrounding the user 110 while the user 110 is performing the activity. In some embodiments, GPS sensors monitor the real-time location of the user 110. In some embodiments, the temperature sensors detect a skin temperature of the user 110. In some embodiments, the IMUs detect angular rate, force, magnetic field, and/or changes in acceleration of the user 110. In some embodiments, the wear detectors determine whether the head-wearable device 130 is being worn by the user 110. In some embodiments, the SpO2 sensors measure blood oxygen saturation of the user 110.

In some embodiments, while the user 110 is performing the activity, the plurality of sensors 140 monitors a plurality of health metrics of the user 110. In some embodiments, a metric pertaining to an attribute of the body of the user 110 (e.g., gait, head position, vertical oscillation, heart rate, breathing rate, pupil dilation, cardiovascular strain, blood oxygen saturation, skin temperature, etc.) is determined based on data from the plurality of sensors 140. In some embodiments, an expected metric is compared to the metric pertaining to the attribute of the body of the user 110, and if a difference between the expected metric and the metric satisfies a predetermined threshold (e.g., is above, below, or equal to the predetermined threshold), an indication is presented pertaining to the attribute of the body of the user 110 at the head-wearable device 130 and/or another device. In some embodiments, the indication is presented at one or more displays of the head-wearable device 130 and/or the other device (e.g., a display of a wrist-wearable device communicatively coupled to the head-wearable device 130). In some embodiments, the indication is a comparative indicator, wherein a current value of the metric and the expected value of the metric are displayed and the user 110 is encouraged to align the current value of the metric and the expected value of the metric. For example, if the user 110 is running and the vertical oscillation is above 3 cm or below-3 cm from a baseline level of 0 cm of vertical oscillation, the display of the head-wearable device can present a range from 3 cm to-3 cm and the current vertical oscillation of the user 110 while the user 110 is running. In some embodiments, the indication is presented at one or more speakers of the head-wearable device 130 and/or the other device. In some embodiments, the indication is presented via haptic feedback at the head-wearable device 130 and/or the other device.

In some embodiments, the altimeter is sampled at approximately 10 Hz. In some embodiments, the IMUs are sampled at approximately 104 Hz. In some embodiments, signal processing algorithms are applied to the altimeter data to extract vertical motion information. In some embodiments, the signal processing includes a high-pass filter combined with peak and valley detection algorithms. In some embodiments, the high-pass filter is used to filter out slopes for vertical oscillation estimation. In some embodiments, a low-pass filter is used for elevation-change estimation. In some embodiments, noise reduction and normalization are applied to the raw altimeter data. In some embodiments, the low-pass filtered signal is differentiated to identify transitions and peaks corresponding to vertical displacement events. In some embodiments, vertical oscillation metrics are determined for various physical activities. In some embodiments, the ideal vertical oscillation for running is 6-8 centimeters per step. In some embodiments, the ideal vertical oscillation for golf is 2-10 centimeters per swing. In some embodiments, the ideal vertical oscillation for rowing is 2-4 centimeters per stroke. In some embodiments, for weightlifting exercises such as squats, pullups, and pushups, the ideal vertical motion is the same depth each repetition. In some embodiments, for activities such as skiing, snowboarding, cycling, equestrian sports, and archery, the ideal vertical motion is as little as possible. In some embodiments, feedback is provided to the user 110 when the vertical oscillation deviates from the ideal range for the particular activity.

In some embodiments, the head-wearable device 130 is used for VR fitness training. In some embodiments, the sensor data from the plurality of sensors 140 including heart rate via iPPG, pupil dilation, microphone recordings of breathing patterns, body movement tracking, and activity-type recognition are analyzed while the user 110 exercises in VR. In some embodiments, personalized training programs are created that adjust in real time based on biometric feedback of the user 110. In some embodiments, if heart rate data indicates suboptimal exertion levels, the VR system automatically intensifies the workout. In some embodiments, real-time feedback on performance and safety are presented to the user 110. In some embodiments, the sensor data is continuously monitored for signs of health risks and the training regimen is adjusted accordingly.

In some embodiments, the head-wearable device 130 includes eye-tracking (ET) cameras and face-tracking (FT) cameras that monitor heart rate. In some embodiments, the IMUs assess overall activity levels and help refine photoplethysmography (PPG) data by reducing noise. In some embodiments, microphones track respiratory fitness. In some embodiments, body-tracking sensors based on ultrasound or radar monitor posture, enhancing the effectiveness of fitness routines. In some embodiments, iPPG, which is a non-invasive method used to detect blood volume changes in the microvascular bed of tissue, is used to track fitness data of the user 110. In some embodiments, rapid fluctuations in the iPPG signal correspond to the heartbeat, reflecting each cardiac cycle's impact on blood volume. In some embodiments, slower fluctuations in the iPPG signal are related to breathing due to respiratory induced amplitude variations (RIAVs). In some embodiments, the chest expansion and contraction with inhalation and exhalation affects intrathoracic pressure and cardiac output, modulating blood flow dynamics and influencing the amplitude of the iPPG waveform. In some embodiments, the iPPG signal captures both cardiac and respiratory rhythms.

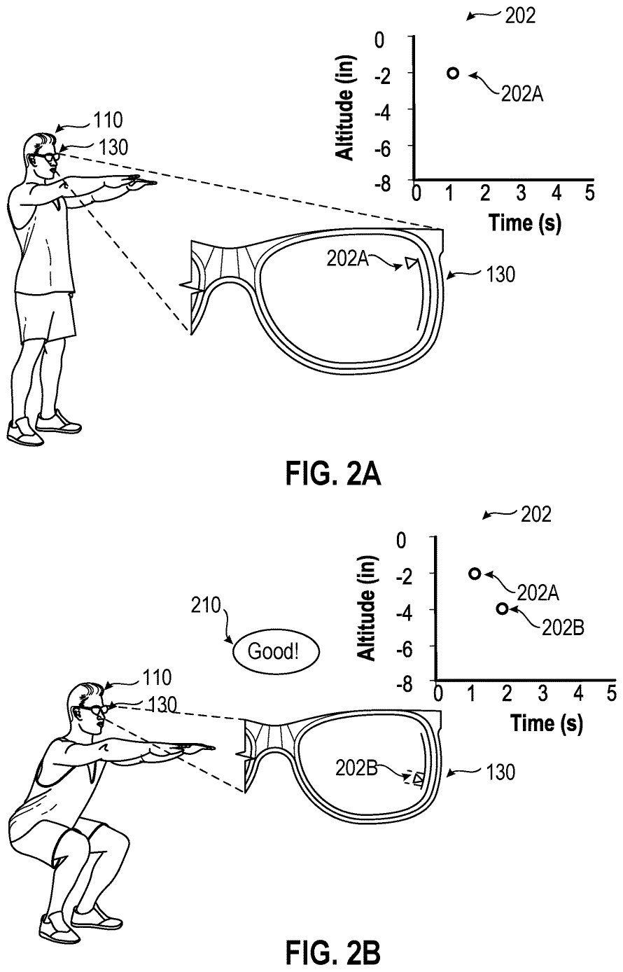

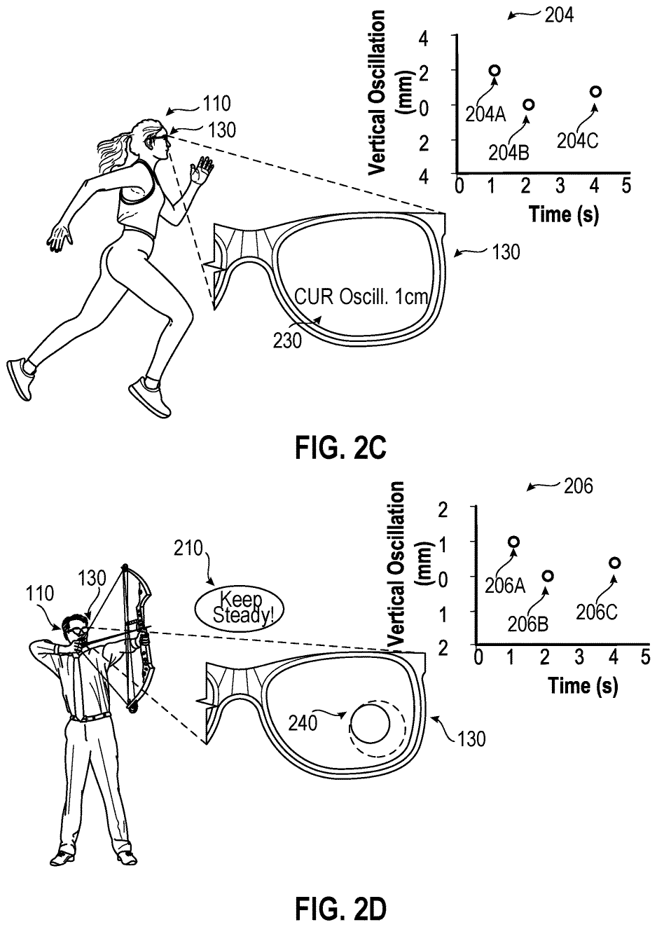

FIGS. 2A-2D illustrate the user 110 wearing the head-wearable device 130 while exercising, in accordance with some embodiments. In some applications, the head-wearable device 130 can utilize an altimeter (in addition to other sensors) to provide feedback or guidance to the user 110 during a fitness activity. For example, the head-wearable device 130 can provide feedback based on altitude regarding a desired range of motion (as depicted in FIGS. 2A and 2B), steadiness (as depicted in FIGS. 2C and 2D), or other suitable applications (stairs climbed, feet descended along a slope, etc.).

FIGS. 2A and 2B illustrate the user 110 performing squats while wearing the head-wearable device 130, in accordance with some embodiments. In some embodiments, while the user 110 is performing squats, the altimeter of the head-wearable device 130 collects altitude data indicative of an altitude of the head of the user 110, and an altitude metric approximating the altitude of the head of the user 110 based on the altitude data is determined, represented as graph 202. In some embodiments, the user 110 is determined to be performing a squat exercise based on the altitude metric and data from one or more of the positional sensors and the IMUs. In some embodiments, a motion profile for performance of the squat exercise is determined based on the data collected by the plurality of sensors 140 as described herein. In some embodiments, the motion profile for the squat exercise requires that the altitude metric reach a particular terminal altitude from a starting altitude, represented by point 202A, corresponding to a midpoint in the squat exercise (e.g., the lowest point of the squat), represented by point 202B. In some embodiments, the user 110 is determined to be performing the squat exercise in accordance with the motion profile by determining whether a difference between the altitude metric and the particular terminal altitude satisfies an acceptable deviation threshold. In some embodiments, responsive to determining that the difference satisfies the acceptable deviation threshold, an indication 210 (e.g., the one or more speakers of the head-wearable device 130 providing “Good!”) is presented pertaining to the squat depth of the user 110 at the head-wearable device 130 and/or the other device. In some embodiments, the indication indicates whether the user 110 is achieving proper squat depth in accordance with the motion profile. In some embodiments, the head-wearable device 130 presents real-time feedback on the altitude of the user 110. For example, the display of the head-wearable device 130 representing points 202A and 202B as a highest and lowest point of a range on the display.

In some embodiments, while the user 110 is performing squats, the positional sensors and IMUs of the head-wearable device 130 collect head position data indicative of a head position of the user 110. In some embodiments, a head position metric is determined based on the head position data. In some embodiments, the motion profile for the squat exercise requires that the head position remain substantially level throughout the squat. In some embodiments, the user 110 is determined to be performing the squat exercise in accordance with the motion profile by determining whether a difference between the head position metric and an expected head position metric satisfies an acceptable deviation threshold. In some embodiments, the head position metric is captured by the altimeter as a vertical oscillation metric. In some embodiments, responsive to determining that the difference satisfies the acceptable deviation threshold (e.g., the head of the user 110 is tilted forward or backward beyond the acceptable deviation threshold), an indication is presented instructing the user 110 to adjust their head position at one or more of the head-wearable device 130 and the other device.

FIG. 2C illustrates the user 110 running while wearing the head-wearable device 130, in accordance with some embodiments. In some embodiments, while the user 110 is running, an altimeter of the head-wearable device 130 collects altitude data indicative of an altitude of the head of the user. In some embodiments, an altitude metric approximating the altitude of the head of the user 110 based on the altitude data is determined. In some embodiments, a determination that the user 110 is performing a running exercise is based on the altitude metric and data from one or more of the gait sensors, the IMUs, and the GPS sensors. In some embodiments, the user 110 voluntarily provides that they are performing a running exercise. In some embodiments, the user 110 voluntarily provides that they are performing a walking exercise, and then begins running, which is detected by the plurality of sensors 140, based on data from the gait sensors, a speedometer, and/or the GPS sensors. In some embodiments, a motion profile for performance of the running exercise is determined, and the motion profile for the running exercise requires that the altitude metric oscillates according to a particular vertical oscillation. For example, whether the user 110 is performing a running exercise may require that a difference between a vertical oscillation indicated by the altitude metric and the particular vertical oscillation satisfies an acceptable deviation threshold. In some embodiments, responsive to determining that the difference satisfies the acceptable deviation threshold, an indication 230 (e.g., “CUR Oscill. 1 cm”) is presented pertaining to the vertical oscillation of the user 110, as represented by graph 204. In some embodiments, the indication 230 indicates whether the user 110 is performing the running exercise in accordance with the motion profile, and is tracked over time as represented by points 204A, 204B, and 204C. In some embodiments, the indication 230 is presented visually on one or more displays, audibly via one or more speakers, and/or via haptic feedback of one or more of the head-wearable device 130 and the other device.

In some embodiments, while the user 110 is running, the heart rate sensors of the head-wearable device 130 collect heart rate data indicative of a heart rate of the user 110, and a heart rate metric is determined based on the heart rate data. In some embodiments, while the user 110 is running, the heart rate metric is compared to an expected heart rate metric, and in accordance with a difference between the heart rate metric and the expected heart rate metric satisfying a predetermined threshold, an indication pertaining to the heart rate of the user 110 is presented. In some embodiments, the eye-tracking sensors capture pupil dilation data, and a cardiovascular strain metric based on the pupil dilation data is determined. In some embodiments, the microphones capture breathing data, and a respiratory efficiency metric based on the breathing data is determined. In some embodiments, the GPS sensors track the location of the user 110, and a pace metric based on the GPS data and a time elapsed during the running exercise is determined.

In some embodiments, the user 110 performs lunges while wearing the head-wearable device 130. In some embodiments, while the user 110 is performing lunges, an altitude metric is calculated based on altitude data collected by the altimeter, a gait metric is calculated based on gait data collected by the gait sensors, and an acceleration metric is calculated based on acceleration data collected by the IMUs. In some embodiments, the user 110 is determined to have successfully performed a lunge exercise based on one or more of the altitude metric, the gait metric, and the acceleration metric. In some embodiments, a motion profile for performance of the lunge exercise is determined or otherwise stored on the head-wearable device 130. In some embodiments, the motion profile for the lunge exercise requires that the altitude metric reach a particular terminal altitude corresponding to a midpoint in the lunge exercise and the gait metric reach a particular stepping distance. In some embodiments, a certain acceleration threshold must be reached by the acceleration metric for the user 110 to be considered to have completed a lunge exercise. In some embodiments, the differences between metrics and expected metrics of any exercise must satisfy acceptable deviation threshold for the exercise to be considered completed by the user 110. In some embodiments, responsive to determining that any of the differences satisfy any of the respective acceptable deviation thresholds, the exercise is considered completed by the user 110. In some embodiments, in accordance with any number of the differences satisfying any of the respective acceptable deviation thresholds, an indication of the lunge form of the user 110 is presented at one or more of the head-wearable device 130 and the other device. For example, if the user 110 achieves an altitude metric and a gait metric indicative of a lunge exercise, but not the required acceleration metric, the display of the head-wearable device 130 can present an encouraging message (e.g., “next time, go faster, you can do this!”) to the user 110.

In some embodiments, the user 110 rides a bicycle while wearing the head-wearable device 130. In some embodiments, while the user 110 is cycling, an altitude metric is determined based on altitude data collected by the altimeter, a heart rate metric is determined based on heart rate data collected by the heart rate sensors, a location is determined based on location data collected by the GPS sensors, and a motion metric is determined based on motion data collected by the IMUs. In some embodiments, a motion profile for the cycling exercise requires that the altitude metric remain substantially constant (e.g., the head of the user 110 does not bounce excessively and/or beyond a certain threshold range while cycling). In some embodiments, the user 110 is determined to be performing the cycling exercise in accordance with the motion profile by determining whether a difference between the altitude metric and a previous altitude metric based on previous altitude data satisfies an acceptable deviation threshold. In some embodiments, responsive to determining that the difference satisfies the acceptable deviation threshold, the user 110 is presented with an indication comprising a cycling form of the user 110 at one or more of the head-wearable device and the other device. In some embodiments, the indication includes a cadence metric determined based on the motion data from the IMUs corresponding to the cycling cadence of the user 110. For example, the display of the head-wearable device 130 can present an indication comprising an acceptable range for the head of the user 110 to be positioned in as well as a rate of speed of the user 110 based on the motion data and/or the location of the user 110.

FIG. 2D illustrates the user 110 using a bow and arrow while wearing the head-wearable device 130, in accordance with some embodiments. In some embodiments, while the user 110 is using the bow and arrow, the altimeter of the head-wearable device 130 collects altitude data corresponding to an altitude of the head of the user 110, and the head-wearable device 130 determines an altitude metric approximating the vertical oscillation of the head of the user 110 based on the altitude data. In some embodiments, a motion profile is determined for performance of the archery exercise based on data from the IMUs, the eye-tracking sensors, and the altitude sensor. In some embodiments, the motion profile for the archery exercise requires that the altitude metric remain substantially constant throughout the draw and release phases of the archery exercise (e.g., the head of the user 110 should not bob or dip during the archery sequence). In some embodiments, the user 110 is determined to be performing the archery exercise in accordance with the motion profile by determining whether a difference between the vertical oscillation and a previous vertical oscillation based on the altitude data satisfies an acceptable deviation threshold. In some embodiments, the altitude data corresponds to the vertical oscillation of the head of the user 110, represented by graph 206 and points 206A, 206B, and 206C. In some embodiments, in accordance with determining the user 110 is performing an archery exercise, a stability indication 240 pertaining to a head stability of the user 110 is presented at one or more of the head-wearable device and other device. In some embodiments, the stability indication 240 is a small circle representing the vertical oscillation of the user 110 within a larger circle indicating the acceptable threshold for the position of the head of the user 110. In some embodiments, the indication 210 (e.g., “Keep steady!) is presented in accordance with the user 110 being at or surpassing the acceptable threshold for the position of the head of the user 110.

In some embodiments, the altimeter and/or the IMUs can provide confirmation of a fall or crash. In some embodiments, IMUs can sense impact and unusual motion, but athletic movements like jumps, sharp stops, or curb drops can cause false positives when detecting falls or crashes, and a specific motion pattern (e.g., a rapid acceleration signal and no movement) can involve a high latency for fall detection. An altimeter can help confirm a fall by detecting a rapid drop in elevation followed by no vertical movement with low latency.

In some embodiments, the altimeter can be used to detect submersion in water. Measured pressure from water immersion is distinct and higher than measured pressure in air (e.g., gauge pressure). In some embodiments, the altitude data can detect when the head-wearable device 130 is submerged and proactively notify the user 110 (e.g., via audio prompts, app notifications, haptic feedback, etc.) that water may have entered the head-wearable device 130 and provide steps for drying the head-wearable device 130. In some embodiments, in accordance with the altitude data indicating that the head-wearable device 130 is no longer submerged, a water rejection sequence (e.g., causing presentation of audio tones from one or more speakers of the head-wearable device 130 to clear water from the head-wearable device 130) is triggered.

In some embodiments, the altimeter both provides accurate estimation of absolute elevation and elevation gain/decline during fitness activity. In some embodiments, the altimeter additionally provides vertical oscillation measurements, tracking the up-and-down bouncing motion of the center of mass of the body of the user 110 during movement. Examples of fitness activities that involve vertical oscillation include: running, where vertical bounce per step is assessed to calculate running efficiency and injury risk, cycling, where head “bobbing” is monitored to ensure maximum power transfer to pedals and to reduce saddle discomfort, golfing, where vertical head movement is tracked during all portions of a swing (e.g., backswing, downswing, and post-impact) and a form of the user 110 is analyzed, skiing and/or snowboarding, where tracking vertical head motion indicates terrain absorption, technique quality, and control, rowing, where there is major full-body movement but minimal vertical oscillation, equestrian (e.g., horseback riding), where there are distinct oscillation patterns with larger magnitudes than other oscillation-dominant activities (e.g., walking or running), and sport shooting (e.g., archery), where there is no elevation change and minimal vertical oscillation. Examples of fitness activities that involve elevation changes include: hiking, which is differentiated from walking using elevation changes, mountain biking, which is differentiated from road biking using elevation changes, trail running, which is differentiated from road running using elevation changes, rock climbing and/or bouldering, where distinct elevation gains and declines occur over time compared to other activities (e.g., by a number of attempted ascents and descents, and elevation scaled over time), sailing, where vertical motion of a boat helps differentiate from other activities, surfing, where vertical motion of waves helps differentiate from other activities (e.g., by tracking a number of waves successfully surfed and time spent paddling), ultimate frisbee, where unique patterns of certain activities (e.g., running, pausing, and jumping) helps differentiate from other sports, various lifting exercises (e.g., squats, pushups, pullups) where vertical motion helps differentiate lifting exercises by accounting for vertical oscillation patterns across exercises, volleyball (e.g., court volleyball or beach volleyball) where there are unique motion patterns (e.g., jumps, dives, squats, etc.), and skateboarding, where there are unique elevation changes caused by various tricks and features (e.g., halfpipes, banks, mounds, etc.). In some embodiments, based on one or more of motion data, elevation data and vertical oscillation data, the head-wearable device 130 predicts what activity the user 110 is performing. For example, if the user 110 wearing head-wearable device 130 is running, jumping, and pausing motion, the vertical oscillation of the user 110 being minimal while the motion of the user 110 is paused, the head-wearable device 130 predicts that the user 110 is playing ultimate frisbee, and provides one or more of real-time and post-activity feedback to the user 110 regarding the activity.

### Providing Real-Time Fitness Feedback Using Multiple Sensors on a Head-Wearable Device

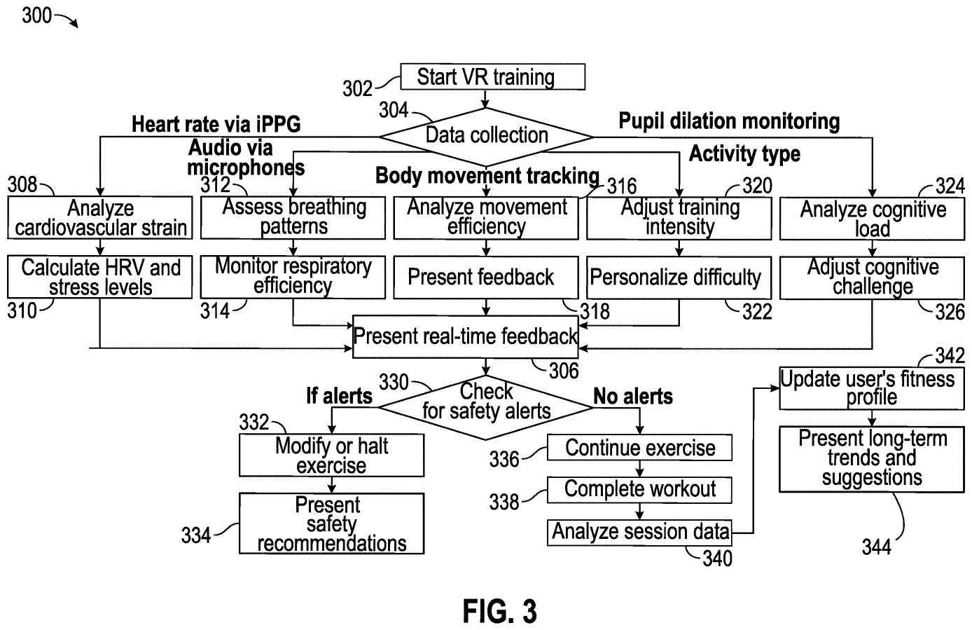

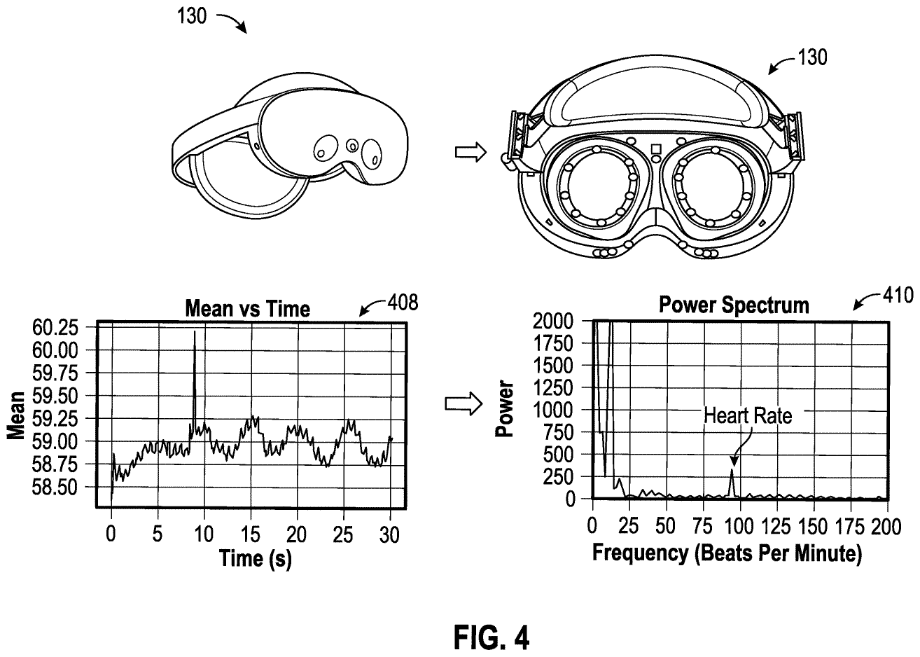

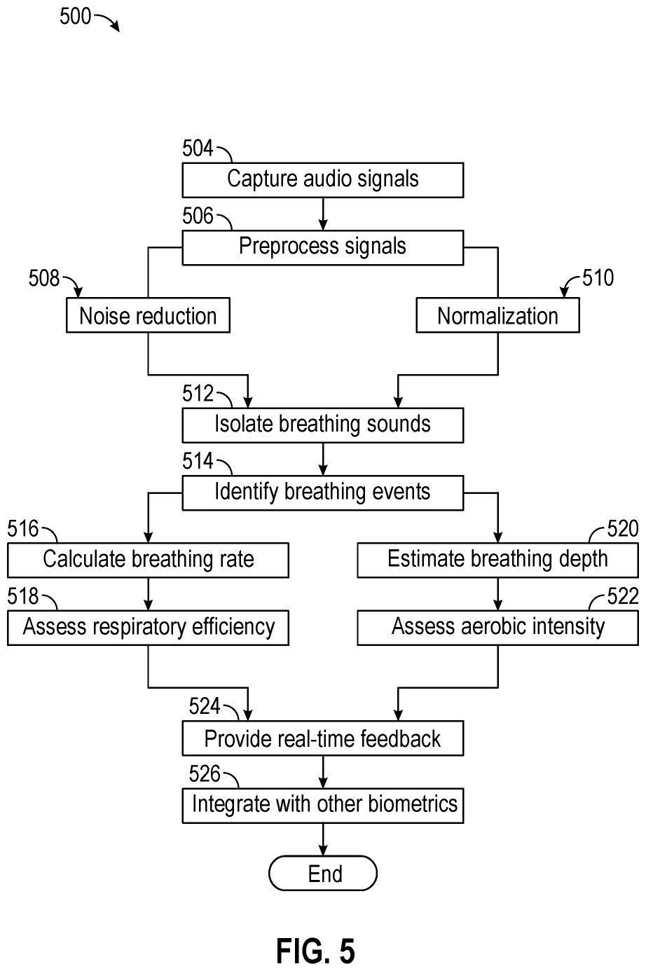

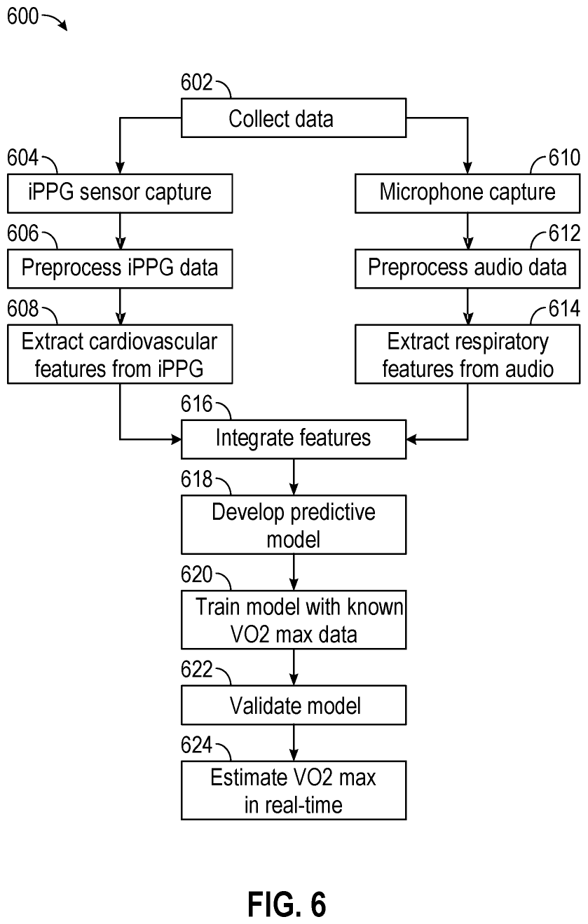

In some applications, the head-wearable device 130 uses the altimeter data along with data from other sensors to provide real-time feedback to the user 110 while the user 110 is exercising, and can adjust a planned fitness routine in accordance with an analysis of the data collected from the sensors on the head-wearable device 130. For example, the head-wearable device 130 can both present real-time feedback to the user 110 and check for safety alerts based on collected data (as depicted in FIG. 3), monitor and assess cardiovascular and respiratory health (as depicted in FIG. 4), identify and isolate breathing events from the user 110 to assess respiratory efficiency and aerobic intensity (as depicted in FIG. 5), estimate VO₂ MAX based on a predictive model (as depicted in FIG. 6), or other suitable applications and/or combination of applications. FIG. 3 shows an example method flow chart for the user 110 wearing the head-wearable device 130 with the plurality of sensors 140 while exercising, in accordance with some embodiments. Operations (e.g., steps) of the method 300 can be performed by one or more processors (e.g., central processing unit and/or MCU) of an XR system. At least some of the operations shown in FIG. 3 correspond to instructions stored in a computer memory or computer-readable storage medium (e.g., storage, RAM, and/or memory) of an XR system. Operations of the method 300 can be performed by a single device alone or in conjunction with one or more processors and/or hardware components of another communicatively coupled device (e.g., a head-wearable device) and/or instructions stored in memory or computer-readable medium of the other device communicatively coupled to the XR system. In some embodiments, the various operations of the methods described herein are interchangeable and/or optional, and respective operations of the methods are performed by any of the aforementioned devices, systems, or combination of devices and/or systems. For convenience, the method operations will be described below as being performed by a particular component or device, but should not be construed as limiting the performance of the operation to the particular device in all embodiments.

The method 300 includes, starting (302) a VR training, collecting (304) data from sensors (e.g., heart rate sensors, an inward-facing camera tracking pupil dilation, audio data captured by one or more microphones, and body movement sensors). In some embodiments, the activity type of an exercise is collected. The method 300 further includes, analyzing (308) cardiovascular strain of the user 110 based on heart rate data and calculating (310) HRV and stress levels of the user 110 based on the analyzed cardiovascular strain of the user 110. The method 300 further includes, assessing (312) breathing patterns of the user 110 based on the audio data captured by the one or more microphones and monitoring (314) the respiratory efficiency of the user 110. The method 300 further includes, analyzing (316) movement efficiency of the user 110 and presenting (318) feedback (e.g., the feedback being targeted at the form and/or technique of the user 110 as they exercise). The method 300 further includes, adjusting (320) the training intensity of the exercise the user 110 is performing and personalizing (322) a difficulty of the exercise based on the adjusted training intensity. In some embodiments, the adjusted training intensity is based on the HRV, stress level, respiratory efficiency, movement efficiency, and any other appropriate metrics of the physiological state of the user 110. The method 300 further includes analyzing (324) the cognitive load of the user 110 and adjusting (326) the cognitive challenge of the user 110 based on the analyzed cognitive load. In some embodiments, adjusting the cognitive challenge is equivalent to personalizing the difficulty of the exercise. The method 300 further includes, based on the calculated HRV and stress, monitored respiratory efficiency, provided feedback, personalized difficulty, and/or adjusted cognitive challenges, presenting (306) real-time feedback to the user 110. In some embodiments, the real-time feedback is presented as an indication at one or more speakers, one or more displays, and/or one or more haptic actuators of the head-wearable device 130 and/or the other device.

The method 300 further includes, checking (330) for safety alerts based on the real-time feedback. In some embodiments, the safety alerts are based on the analyzed and assessed data collected while the user 110 exercises. If it is determined that a safety alert should be provided, modifying or halting (332) the exercise, the modifying and halting based on the severity of the alert, and if the exercise is halted, presenting (334) safety recommendations to the user 110. For example, if the user 110 has an elevated heart rate, the exercise is halted and the user 110 is instructed to take deep breaths to slow their heart rate.

In some embodiments, the safety alerts include abnormal heart rate notifications for high or low heart rates. In some embodiments, the safety alerts include arrhythmia detection. In some embodiments, the safety alerts include respiratory alerts for rapid or shallow breathing. In some embodiments, the safety alerts include warnings for excessive fatigue. In some embodiments, the safety alerts include dehydration risk warnings. In some embodiments, the safety alerts include overheating warnings. In some embodiments, the safety alerts include alerts for poor posture or incorrect form. In some embodiments, the safety alerts include insufficient recovery warnings. In some embodiments, the safety alerts include unsafe environmental conditions warnings. In some embodiments, the safety alerts include equipment malfunction warnings.

The method 300 further includes, if it is determined that no alert should be presented, continuing (336) the exercise until the workout is completed (338), and analyzing (340) workout session data, including the calculated HRV and stress, monitored respiratory efficiency, provided feedback, personalized difficulty, and/or adjusted cognitive challenges. The method 300 further includes updating (342) the user's fitness profile based on the analyzed session data, and presenting (344) long-term trends and suggestions based on the updated fitness profile and the analyzed session data.

In some applications, the head-wearable device can utilize non-invasive methods of tracking the skin of the user 110 near an eye of the user 110 to monitor and assess cardiovascular and respiratory health efficiency while the user 110 is exercising, and can be integrated and/or cross-referenced with other collected biometrics to provide real-time feedback to the user 110, as described herein. FIG. 4 illustrates data collected from the head-wearable device 130 with the plurality of sensors 140 while the user 110 wearing the head-wearable device 130 exercises, in accordance with some embodiments. In imaging photoplethysmography (iPPG), which is a non-invasive method used to detect blood volume changes in the microvascular bed of tissue, breathing-related changes in blood oxygenation may be observed alongside heartbeat-related fluctuations. The rapid fluctuations in the iPPG signal correspond to the heartbeat, reflecting each cardiac cycle's impact on blood volume. In contrast, slower fluctuations are related to breathing. These are due to RIAVs, which occur as the chest expands and contracts with inhalation and exhalation, affecting intrathoracic pressure and cardiac output. This modulation of blood flow dynamics influences the amplitude of the iPPG waveform, allowing the signal to capture both cardiac and respiratory rhythms. These patterns may be used to monitor and assess cardiovascular and respiratory health effectively.

As shown in FIG. 4, the head-wearable device 130 equipped with eye tracking cameras may capture IR images of the skin of the user 110 near an eye of the user 110. An iPPG signal 408 (e.g., mean vs time) may be developed from these images. Subjecting this signal 408 to a Fourier transform may find a heart rate 410 (e.g., power vs frequency (e.g., beats per minute) of the user 110). This heart rate 410 may be monitored over time to determine variability (e.g., HRV) in the heart rate 410.

In some applications, audio signals captured by microphones included with the head-wearable device 130 can be processed such that individual breathing events are analyzed to monitor and assess the respiratory efficiency and aerobic intensity of the user 110 as the user 110 is exercising, and can be integrated and/or cross-referenced with other collected biometrics to provide real-time feedback to the user 110, as described herein. FIG. 5 shows an example method flow chart for the user 110 wearing the head-wearable device 130 with the plurality of sensors 140 while exercising, in accordance with some embodiments. Operations (e.g., steps) of the method 500 can be performed by one or more processors (e.g., central processing unit and/or MCU) of an XR system. At least some of the operations shown in FIG. 5 correspond to instructions stored in a computer memory or computer-readable storage medium (e.g., storage, RAM, and/or memory) of an XR system. Operations of the method 500 can be performed by a single device alone or in conjunction with one or more processors and/or hardware components of another communicatively coupled device (e.g., a head-wearable device) and/or instructions stored in memory or computer-readable medium of the other device communicatively coupled to the XR system. In some embodiments, the various operations of the methods described herein are interchangeable and/or optional, and respective operations of the methods are performed by any of the aforementioned devices, systems, or combination of devices and/or systems. For convenience, the method operations will be described below as being performed by a particular component or device, but should not be construed as limiting the performance of the operation to the particular device in all embodiments.

The method 500 includes capturing (504) audio signals using one or more microphones of the head-wearable device 130, preprocessing (506) the signals to isolate for breathing patterns of the user 110, the preprocessing including one or more of reducing (508) the noise in the audio signals and normalizing (510) the audio signals. The method 500 further includes isolating (512) the breathing sounds of the preprocessed audio signals and identifying (514) individual breathing events (e.g., inhale and exhale cycles) of the user 110. The method 500 further includes one or more of calculating (516) a breathing rate of the user 110 (e.g., how often the user 110 inhales and exhales) and assessing (518) a respiratory efficiency of the user 110 based on the calculated breathing rate, and estimating (520) a breathing depth of the user 110 (e.g., how long each inhale and exhale cycle occurs for) and assessing (522) an aerobic intensity of the user 110 based on the estimated breathing depth. In some embodiments, the breathing rate is calculated by counting breaths per minute, and the depth of each breath is estimated by analyzing the amplitude and duration of inhalation and exhalation sounds. The method 500 further includes providing (524) real-time feedback to the user 110 based on the assessed respiratory efficiency and assessed aerobic intensity (e.g., “You are hyperventilating, reduce the intensity of your exercise”) and integrating (526) the real-time feedback with other biometrics of the user 110.

In some implementations, calculation of breathing rate from audio signals may include event detection and counting breaths. For example, the event detection may include identifying the start of each inhalation and the end of each exhalation by detecting changes in signal amplitude or frequency. Also, the counting of breaths may include counting the number of complete breath cycles over a known period. In this context, if n breaths are counted over t seconds, the breathing rate (BR) in breaths per minute may be calculated as: BR=(n/t)*60.

In some implementations, estimating breathing depth from audio signals may include amplitude and duration analysis and calibration. For example, amplitude and duration may be analyzed by analyzing peaks amplitudes and durations of inhalations. Then, using known volume measurements for calibration, a breathing depth (e.g., volume) may be estimated as: V=a*A+b*D+c, where V is the estimated volume (e.g., breathing depth), A is the amplitude, D is the duration, and a, b, and c are coefficients determined during calibration. Variations in breathing rate and depth in response to different levels of physical activity may provide insights into an individual's aerobic capacity and fitness level.

### Calculating Vo₂ Max Using iPPG Sensors on a Head-Wearable Device

In some embodiments, aerobic capacity and ventilation, breathing patterns and fitness levels, and predictive value of breathing rate are measured by the plurality of sensors 140. For example, aerobic capacity, often measured as VO₂ MAX (e.g., the maximum rate of oxygen consumption), is closely linked to ventilatory responses during exercise. As exercise intensity increases, so does the demand for oxygen, leading to increases in both the rate and depth of breathing (e.g., tidal volume and respiratory rate). This relationship is foundational in exercise physiology. Additionally, studies have shown that more aerobically fit individuals often have more efficient breathing patterns, characterized by greater tidal volumes and lower respiratory rates at a given submaximal exercise intensity. This efficiency reflects a better-developed respiratory system and cardiovascular fitness. Also, research has explored using breathing rate as a non-invasive predictor of aerobic fitness. For example, the ventilatory threshold, which is the point during incremental exercise at which ventilation starts to increase at a faster rate than VO₂, is a marker of aerobic performance and may be detected through changes in breathing patterns.

FIG. 6 shows an example method flow chart for the user 110 wearing the head-wearable device 130 with the plurality of sensors 140 while exercising, in accordance with some embodiments. Operations (e.g., steps) of the method 600 can be performed by one or more processors (e.g., central processing unit and/or MCU) of an XR system. At least some of the operations shown in FIG. 6 correspond to instructions stored in a computer memory or computer-readable storage medium (e.g., storage, RAM, and/or memory) of an XR system. Operations of the method 600 can be performed by a single device alone or in conjunction with one or more processors and/or hardware components of another communicatively coupled device (e.g., a head-wearable device) and/or instructions stored in memory or computer-readable medium of the other device communicatively coupled to the XR system. In some embodiments, the various operations of the methods described herein are interchangeable and/or optional, and respective operations of the methods are performed by any of the aforementioned devices, systems, or combination of devices and/or systems. For convenience, the method operations will be described below as being performed by a particular component or device, but should not be construed as limiting the performance of the operation to the particular device in all embodiments.

The method 600 includes collecting (602) data from the plurality of sensors 140 of the head-wearable device 130, capturing (604) iPPG data from iPPG sensors, preprocessing (606) the iPPG data, extracting (608) cardiovascular features from the iPPG data, capturing (610) audio data from one or more microphones, preprocessing (612) the audio data, extracting (614) respiratory features from the audio data, and integrating (616) the extracted cardiovascular features and extracted respiratory features into raw VO₂ data, developing (618) a predictive model to predict VO₂ MAX, training (620) the predictive model with known VO₂ MAX data, validating (622) the trained predictive model, and estimating (624) the VO₂ MAX of the user 110 in real time based on the trained predictive model and the raw VO₂ data. In some embodiments, the iPPG sensors are cameras that capture image data that detect changes in skin color with each heartbeat of the user 110, providing data on heart rate and variability influenced by breathing patterns and exercise intensity. In some embodiments, the one or more microphones capture the sound of the user 110 breathing to record the rate and depth of breaths, analyzing the rhythm and intensity of respiratory activity, as described herein.

In some embodiments, the predictive model may be trained using data from subjects with known VO₂ MAX values and validated at step 622 by comparing its estimates against additional measured values to confirm accuracy.

### Calculating Vo₂ Max Using Pupil Dilation Sensors on a Head-Wearable Device

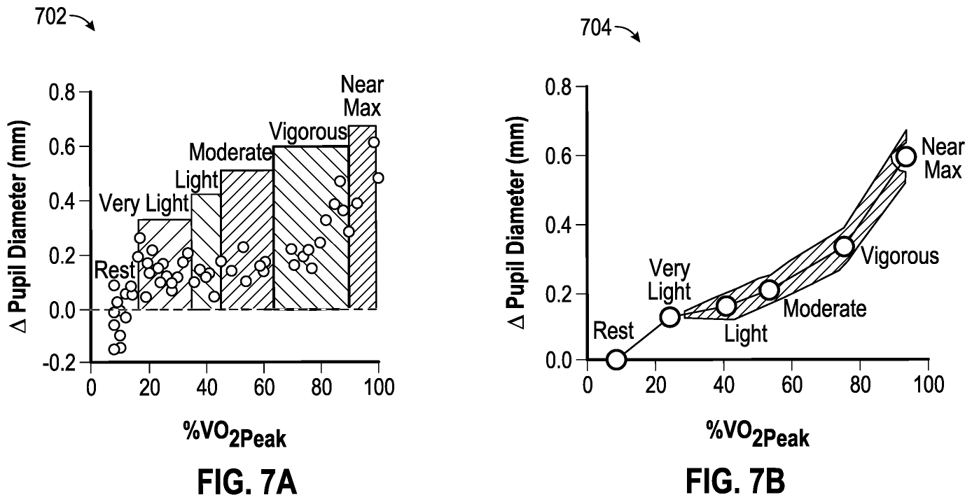

In some applications, the VO₂ MAX of the user 110 is calculated using one or more of the iPPG sensors (as depicted in FIG. 6) and pupil dilation sensors (e.g., inward-facing cameras) to provide real-time feedback to the user 110 of the head-wearable device 130 while the user 110 performs a fitness activity. For example, the head-wearable device 130 can collect both iPPG data and pupil dilation data and cross-validate the analysis of the VO₂ MAX of the user 110 by comparing the analysis of the iPPG data with the analysis of the pupil dilation data. FIGS. 7A and 7B illustrate pupil dilation correlation with VO₂ MAX, in accordance with some embodiments. Graph 702 illustrates change in pupil diameter vs percentage of VO₂ MAX for raw data points, and graph 704 illustrates an extrapolated correlation that may be used to determine VO₂ MAX from change in pupil diameter. Pupil dilation during graded exercise may serve as a physiological indicator of the ascending arousal system activation, particularly noticeable during very-light to light exercise intensities. In some embodiments, the process involves two distinct phases of pupil dilation: an initial increase at very-light exercise intensity, which correlates with psychological arousal, and a more pronounced increase at moderate to maximal exercise intensities, aligning with the pattern of ventilation increase. Pupil dilation may be a useful marker for arousal system activation starting from very-light exercise intensities. Mild physical activities may activate the brain's arousal system, which is essential for cognitive functions and overall mental health. The unique response of pupil dilation at lower exercise intensities may be a non-invasive measure to assess brain arousal states during physical activity. When wearing an AR or VR headset (e.g., head-wearable device 130), confounding factors from ambient light may be eliminated by normalizing for the amount of light that is reaching the eye (e.g., as measured by ambient light sensors in AR or known from the rendering pipeline in VR). In this way, the process illustrated in FIG. 6 may be expanded to include pupil dilation (and the correlation to VO₂ MAX) as one of the inputs into the training and inference pipeline.

In some embodiments, the head-wearable device 130 is configured to filter out natural pupillary micro-fluctuations, commonly referred to as hippus or pupillary unrest, which are inherently synchronized with a user's respiratory and cardiac cycles. During physical activity, raw pupillometry data is received from the one or more inward-facing cameras and is fused with physiological sensor data, such as photoplethysmography (PPG) data or IMU-derived respiration data. By mapping respiratory cycles of the user 110 (e.g., inhale and exhale phases) and/or cardiac cycles, an adaptive filter, such as an adaptive bandpass filter, can be applied to the pupillometry data stream. This adaptive filtering subtracts the cyclical hippus fluctuations, thereby generating a smoothed baseline trend that isolates macro-changes in pupil size indicative of systemic arousal, physical fatigue, or VO₂ MAX levels.

In some embodiments, a pupil size estimation model is configured to isolate autonomic cognitive arousal from mechanical pupillary adjustments (e.g., a “near triad” reflex). When the user 110 focuses on a virtual or physical object in the near-field, the eyes naturally undergo convergence, accommodation, and miosis (pupil constriction). To prevent this mechanical constriction from being misinterpreted as a decrease in arousal or fatigue, a z-depth (virtual or physical distance) of the object the user 110 is currently fixated on is determined by utilizing gaze-tracking data and spatial mapping. An expected mechanical pupil constriction value is calculated based on the determined z-depth. By mathematically subtracting this expected mechanical constriction from the raw measured pupil diameter, vergence-accommodation artifacts are mitigated. This yields a normalized pupil size metric that accurately reflects the purely autonomic arousal state of the user 110, independent of the focal distance of the user 110.

### Determining Emergency Events With Data From a Plurality of Sensors

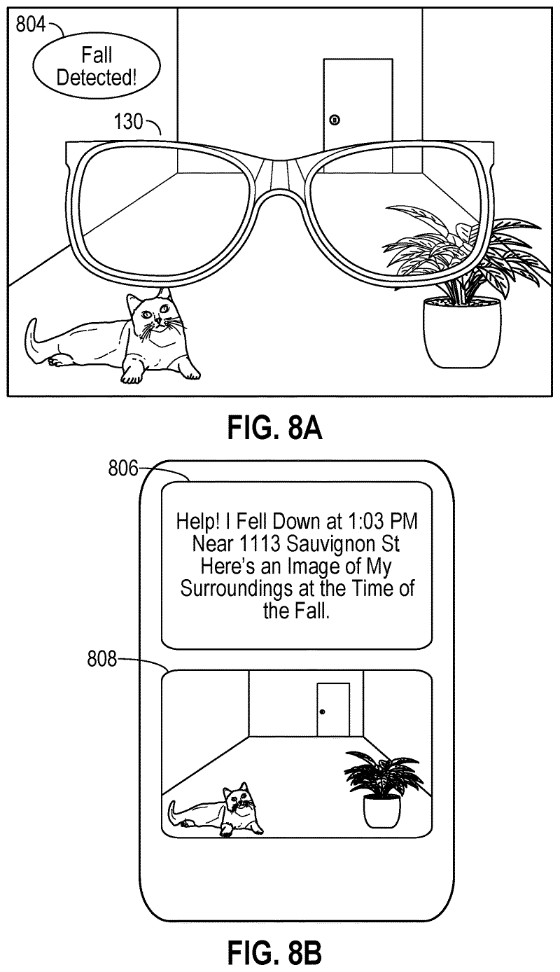

In some applications, the real-time feedback and monitoring performed by the head-wearable device 130 is not limited to or constrained by the user 110 exercising or performing a fitness activity, and can activate in accordance with the user 110 having an emergency that requires rapid or immediate response. FIGS. 8A and 8B illustrate the user 110 wearing the head-wearable device 130 while active, in accordance with some embodiments. FIG. 8A illustrates the user 110 standing while wearing head-wearable device 130, in accordance with some embodiments. In some embodiments, a further metric pertaining to a physiological attribute of the body of the user 110 is determined based on further data collected by one or more of a temperature sensor, the IMUs, an eye-tracking sensor, a wear detector, a camera, and the GPS sensors. In some embodiments, based on the further data, an emergency event is determined to have occurred based on the physiological attribute. For example, if the user 110 rapidly falls to the ground and remains on the ground for a threshold amount of time, the head-wearable device 130 can determine that the user 110 had an accident based on the motion data from the IMUs, image data from the camera, and an elevated temperature captured by the temperature sensor. In some embodiments, an alert 804 (e.g., “Fall detected!”) is presented at the head-wearable device 130. In some embodiments, an emergency action is performed based on the determined emergency event. In some embodiments, the emergency action comprises causing the camera to capture image data 808 of a field of view of the user 110 and sending the image data 808 to one or more of an emergency contact of the user 110 and emergency services (e.g., texting 911 or calling 911 and generating audio data to communicate with an emergency services operator).

FIG. 8B illustrates the user 110 on the ground while wearing head-wearable device 130 after falling, in accordance with some embodiments. In some embodiments, in accordance with the determination that the emergency event has occurred, a message 806 is drafted including the image data 808 of the field of view of the user 110 (e.g., “Help! I fell down at 1:03 PM Near 1113 Sauvignon St Here's an Image of My Surroundings at the Time of the Fall.”). In some embodiments, the message 806 is automatically sent to one or more emergency contacts previously designated by the user 110. In some embodiments, the head-wearable device 130 provides an indication to the user 110 requesting that the user 110 cancel the automatic sending of the message 806 within a threshold amount of time (e.g., 10 seconds). In some embodiments, the message 806 contains GPS coordinates of the user 110. In some embodiments, the user 110 is determined to have fallen based on a rapid change in acceleration at or exceeding an acceleration threshold and/or, after the rapid change in acceleration has occurred, the user 110 remains at a position for a threshold amount of time (e.g., 30 seconds). For example, if the user 110 rapidly falls to the ground and remains on the ground for one minute, the emergency event is determined to have occurred.

In some embodiments, the head-wearable device 130 can implement a multi-modal emergency determination and response system. In some embodiments, outdoor enthusiasts engaged in activities like hiking, trekking, mountaineering, and backcountry skiing face significant safety risks when they become gradually incapacitated. In some embodiments, gradual incapacitation includes hypothermia where core body temperature drops, hyperthermia or heat stroke where core body temperature rises, altitude sickness, injury leading to immobility, and medical emergencies such as diabetic shock and cardiac events. In some embodiments, unlike sudden events such as falls and crashes that can be detected via impact sensors, gradual incapacitation often goes undetected until mitigation is more difficult or requires intervention. In some embodiments, current emergency determination systems focus on sudden impact events and do not address scenarios where a person slowly becomes unable to respond. In some embodiments, in remote outdoor settings, the person may be unconscious or unable to manually trigger an SOS, cell service may be limited requiring satellite-based communication, and first responders need visual context to locate the person in wilderness terrain.

In some embodiments, the head-wearable device 130 integrates a body temperature sensor and leverages existing IMU and accelerometer sensors to implement the multi-modal emergency determination system. In some embodiments, the body temperature sensor is a contact sensor or a non-contact infrared (IR) thermometer. In some embodiments, temperature sensing is performed via a temple or ear contact sensor. In some embodiments, the head-wearable device 130 includes IR temperature sensors positioned on both temple arms of the glasses near the hinge area, enabling non-contact body temperature monitoring from the temple region of the wearer. In some embodiments, the head-wearable device 130 includes IR LED illuminators for providing infrared illumination.

In some embodiments, the sensor data is continuously monitored, the sensor data including temperature sensing to monitor body temperature, motion and activity detection using the IMU and accelerometer to track user movement and activity state, and eye tracking using eye tracking cameras to detect if eyes are closed for a long time, pupil dilation, or no movement. In some embodiments, anomaly detection is performed by combining signals from multiple sensors. In some embodiments, the normal body temperature range is 36.5-37.5° C., and an alert condition is triggered when body temperature is less than 35° C. indicating hypothermia or greater than 39° C. indicating hyperthermia. In some embodiments, the normal eye-tracking state includes normal pupil movement, and an alert condition is triggered when pupil movement is abnormal, there is dilation, or there is no movement. In some embodiments, the normal motion and activity state includes regular movement patterns, and an alert condition is triggered when there is prolonged inactivity exceeding 10-15 minutes with no significant motion. In some embodiments, the normal device position is normal wearing orientation, and an alert condition is triggered when the device is still on the head of the user 110 versus removed or dropped.

### Example Diagrams for Providing Real-Time Fitness Feedback to a User

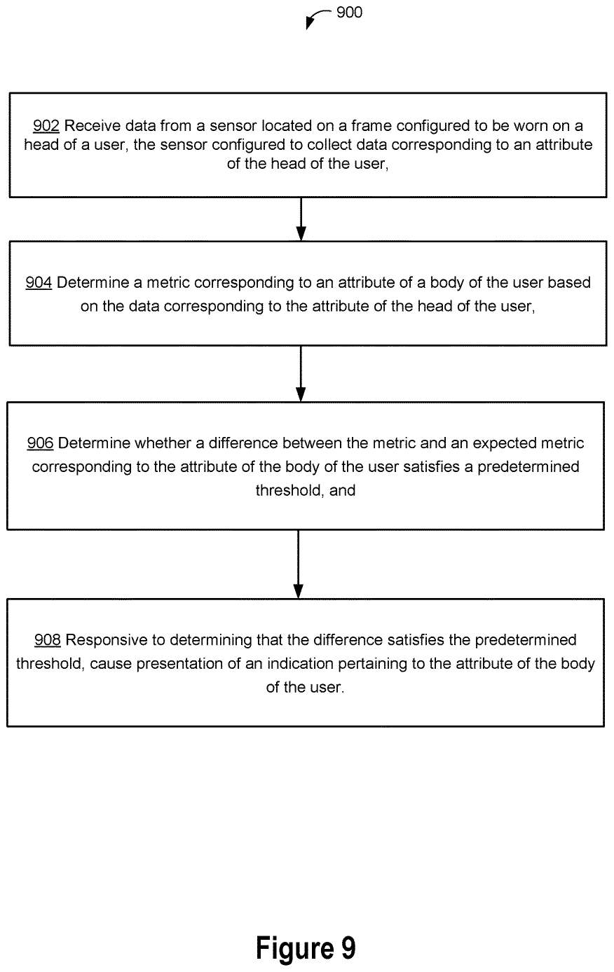

FIG. 9 illustrates a flow diagram of a method of monitoring data while the user 110 exercises, in accordance with some embodiments. Operations (e.g., steps) of the method 900 can be performed by one or more processors (e.g., central processing unit and/or MCU) of an XR system. At least some of the operations shown in FIG. 9 correspond to instructions stored in a computer memory or computer-readable storage medium (e.g., storage, RAM, and/or memory) of an XR system. Operations of the method 900 can be performed by a single device alone or in conjunction with one or more processors and/or hardware components of another communicatively coupled device (e.g., a head-wearable device) and/or instructions stored in memory or computer-readable medium of the other device communicatively coupled to the XR system. In some embodiments, the various operations of the methods described herein are interchangeable and/or optional, and respective operations of the methods are performed by any of the aforementioned devices, systems, or combination of devices and/or systems. For convenience, the method operations will be described below as being performed by a particular component or device, but should not be construed as limiting the performance of the operation to the particular device in all embodiments.

(A1) The method 900 includes receiving (902) data from a sensor located on a frame configured to be worn on a head of a user, the sensor configured to collect data corresponding to an attribute of the head of the user, determining (904) a metric corresponding to an attribute of a body of the user based on the data corresponding to the attribute of the head of the user, determining (906) whether a difference between the metric and an expected metric corresponding to the attribute of the body of the user satisfies a predetermined threshold, and responsive to determining that the difference satisfies the predetermined threshold, causing (908) presentation of an indication pertaining to the attribute of the body of the user. For example, as shown in at least FIGS. 2A and 2B, a user (110) wearing a head-wearable device (130) performs a squat starting at a first point (202A) and finishing at a second point (202B), where the head-wearable device (130) presents an indication (210) (e.g., “Good!”) pertaining to a successful completion of the squat.

(A2) In some embodiments of A1, the method 900 further includes receiving additional data from the sensor, determining that the metric has changed based on the additional data corresponding to the attribute of the head of the user, determining that a second difference between the changed metric and the expected metric pertaining to the attribute of the body of the user does not satisfy the predetermined threshold, and responsive to determining that the second difference does not satisfy the predetermined threshold, causing presentation of a second indication pertaining to the attribute of the user, wherein the second indication indicates that the second difference does not satisfy the predetermined threshold. For example, as shown in FIG. 2D, a user (110) wearing a head-wearable device (130) uses a bow and arrow over three points (206A) (206B) (206C) and an indication (210) (e.g., “Keep Steady!”) is presented at the head-wearable device (130) in accordance with the user (110) not satisfying a predetermined threshold (e.g., keeping the bow steady).

(A3) In some embodiments of any of A1-A2, the method 900 further includes receiving yet additional data from an additional sensor, distinct from the sensor, determining an additional metric, distinct from the metric, corresponding to an additional attribute of the body of the user, distinct from the attribute, based on the yet additional data corresponding to the additional attribute and the data corresponding to the attribute of the head of the user, determining whether an additional difference between the additional metric and an additional expected metric corresponding to the additional attribute of the body of the user satisfies an additional predetermined threshold, and responsive to determining that the additional difference satisfies the additional predetermined threshold, causing presentation of an indication pertaining to the additional attribute of the user. For example, as shown in at least FIG. 3, after starting (302) a VR training, data collecting (304) of multiple metrics (308) (312) (316) (320) (324) occurs and additional differences are calculated and an additional indication is presented (306).

(A4) In some embodiments of any of A1-A3, the sensor comprises an altimeter configured to collect altitude data corresponding to an altitude of the head of the user, determining the metric corresponding to the attribute of the user comprises determining an altitude metric approximating the altitude of the head of the user based on the altitude data, and the method 900 further includes determining that the user is performing a selected exercise based on the altitude metric, determining a motion profile for performance of the selected exercise, determining whether the difference between the metric and the expected metric satisfies the predetermined threshold comprises determining whether the user is performing the selected exercise in accordance with the motion profile based on the altitude metric, and the indication pertaining to the attribute of the user indicates whether the user is performing the selected exercise in accordance with the motion profile. For example, as shown in at least FIGS. 2A and 2B, a user (110) wearing a head-wearable device (130) performs a squat starting at a first point (202A) and finishing at a second point (202B), where the head-wearable device (130) presents an indication (210) (e.g., “Good!”) pertaining to a successful completion of the squat.

(A5) In some embodiments of A4, the motion profile for the selected exercise requires that the altitude metric reach a particular terminal altitude corresponding to a midpoint in the selected exercise, and determining whether the user is performing the selected exercise in accordance with the motion profile comprises determining whether a difference between the altitude metric and the particular terminal altitude satisfies an acceptable deviation threshold. For example, as shown in at least FIGS. 2A and 2B, a user (110) wearing a head-wearable device (130) performs a squat starting at a first point (202A) and finishing at a second point (202B), where the head-wearable device (130) presents an indication (210) (e.g., “Good!”) pertaining to a successful completion of the squat.

(A6) In some embodiments of any of A4-A5, the motion profile for the selected exercise requires that the altitude metric oscillates within a particular vertical oscillation threshold, and determining whether the user is performing the selected exercise in accordance with the motion profile comprises determining whether a vertical oscillation indicated by the altitude metric is maintained within the particular vertical oscillation threshold. For example, as shown in FIG. 2D, a user (110) wearing a head-wearable device (130) uses a bow and arrow over three points (206A) (206B) (206C) and an indication (210) (e.g., “Keep Steady!”) is presented at the head-wearable device (130) in accordance with the user (110) not satisfying a predetermined threshold (e.g., keeping the bow steady).

(A7) In some embodiments of any of A4-A6, the motion profile for the selected exercise requires that the altitude metric have a substantial variation value, and determining whether the user is performing the selected exercise in accordance with the motion profile comprises determining whether a difference between the altitude metric and a previous altitude metric based on the altitude data is at or above the substantial variation value.

(A8) In some embodiments of any of A1-A8, the method 900 further includes determining a performance metric based on a selected exercise and the difference between the metric and the expected metric, and causing presentation of a real-time indication of the performance metric.

(A9) In some embodiments of any of A1-A9, the user is performing a selected exercise, and the method 900 further includes receiving physiological data during a period of time from a plurality of sensors included with the XR device, wherein the physiological data comprises one or more of heart rate data, gait data, and pupil dilation data, receiving action data during the period of time from a second plurality of sensors included with the XR device, distinct from the plurality of sensors, wherein the action data comprises one or more of head position data, audio data, and body position data, determine a plurality of metrics during the period of time, distinct from the metric, corresponding to a plurality of attributes of the body of the user, distinct from the attribute, based on the physiological data and the action data corresponding to the plurality of attributes, during the period of time, determine whether a plurality of differences between the plurality of metrics and a plurality of expected metrics corresponding to the plurality of attributes of the body of the user satisfies a plurality of additional thresholds, and responsive to determining that any of the plurality of differences between the plurality of metrics and the plurality of expected metrics corresponding to the plurality of attributes of the body of the user satisfies a plurality of predetermined thresholds at any point in time during the period of time, cause presentation of an indication instructing the user to alter performance of the selected exercise.

(A10) In some embodiments of A9, responsive to determining that none of the plurality of differences between the plurality of metrics and the plurality of expected metrics corresponding to the plurality of attributes of the body of the user satisfies the plurality of predetermined thresholds, cause presentation of an indication encouraging the user to exert more effort in the exercise, and responsive to determining that any of the plurality of differences between the plurality of metrics and the plurality of expected metrics corresponding to the plurality of attributes of the body of the user satisfies the plurality of predetermined thresholds at any point in time during the period of time, cause presentation of an indication instructing the user to exert less effort in the exercise.

(A11) In some embodiments of any of A1-A10, the method 900 further includes determining a further metric corresponding to a physiological attribute of the body of the user based on the data of the attribute of the body of the user, determining that an emergency event has occurred based on the physiological attribute, and performing an emergency action based on the emergency event. For example, as shown in at least FIGS. 8A-8B, a head-wearable device (130) determines a wearer of the head-wearable device (130) has fallen and causes capture of image data comprising a field of view of the wearer (808) and drafts a message (806) to send to emergency services.

(A12) In some embodiments of A11, the determination that an emergency event has occurred is further based on further data from one or more of a temperature sensor, an inertial measurement unit, an eye-tracking sensor, a wear detector, a camera, and a GPS sensor.

(A13) In some embodiments of any of A11-A12, the further metric is an acceleration metric approximating an acceleration of the head of the user, the emergency event comprises the acceleration metric exceeding an emergency acceleration threshold and the user remaining at a position with zero acceleration for a threshold amount of time, and the emergency action comprises: causing a camera to capture image data of a field of view of the user, and sending the image data to one or more of an emergency contact of the user and emergency services.

(B1) In accordance with some embodiments, a system that includes a wrist-wearable device (or a plurality of wrist-wearable devices) and a pair of augmented-reality glasses, and the system is configured to perform operations corresponding to any of A1-A13.

(C1) In accordance with some embodiments, a non-transitory computer-readable storage medium including instructions that, when executed by a computing device in communication with a pair of augmented-reality glasses, cause the computer device to perform operations corresponding to any of A1-A13.

(D1) In accordance with some embodiments, a method of operating a pair of augmented-reality glasses, including operations that correspond to any of A1-A13.

The devices described above are further detailed below, including wrist-wearable devices, headset devices, systems, and haptic feedback devices. Specific operations described above may occur as a result of specific hardware, such hardware is described in further detail below. The devices described below are not limiting and features on these devices can be removed or additional features can be added to these devices.

### Example Extended-Reality Systems

FIGS. 10A, 10B, 10C-1, and 10C-2 illustrate example XR systems that include AR and MR systems, in accordance with some embodiments. FIG. 10A shows a first XR system 1000a and first example user interactions using a wrist-wearable device 1026, a head-wearable device (e.g., AR device 1028), and/or a HIPD 1042. FIG. 10B shows a second XR system 1000b and second example user interactions using a wrist-wearable device 1026, AR device 1028, and/or an HIPD 1042. FIGS. 10C-1 and 10C-2 show a third MR system 1000c and third example user interactions using a wrist-wearable device 1026, a head-wearable device (e.g., an MR device such as a VR device), and/or an HIPD 1042. As the skilled artisan will appreciate upon reading the descriptions provided herein, the above-example AR and MR systems (described in detail below) can perform various functions and/or operations.

The wrist-wearable device 1026, the head-wearable devices, and/or the HIPD 1042 can communicatively couple via a network 1025 (e.g., cellular, near field, Wi-Fi, personal area network, wireless LAN). Additionally, the wrist-wearable device 1026, the head-wearable device, and/or the HIPD 1042 can also communicatively couple with one or more servers 1030, computers 1040 (e.g., laptops, computers), mobile devices 1050 (e.g., smartphones, tablets), and/or other electronic devices via the network 1025 (e.g., cellular, near field, Wi-Fi, personal area network, wireless LAN). Similarly, a smart textile-based garment, when used, can also communicatively couple with the wrist-wearable device 1026, the head-wearable device(s), the HIPD 1042, the one or more servers 1030, the computers 1040, the mobile devices 1050, and/or other electronic devices via the network 1025 to provide inputs.

Turning to FIG. 10A, a user 1002 is shown wearing the wrist-wearable device 1026 and the AR device 1028 and having the HIPD 1042 on their desk. The wrist-wearable device 1026, the AR device 1028, and the HIPD 1042 facilitate user interaction with an AR environment. In particular, as shown by the first AR system 1000a, the wrist-wearable device 1026, the AR device 1028, and/or the HIPD 1042 cause presentation of one or more avatars 1004, digital representations of contacts 1006, and virtual objects 1008. As discussed below, the user 1002 can interact with the one or more avatars 1004, digital representations of the contacts 1006, and virtual objects 1008 via the wrist-wearable device 1026, the AR device 1028, and/or the HIPD 1042. In addition, the user 1002 is also able to directly view physical objects in the environment, such as a physical table 1029, through transparent lens(es) and waveguide(s) of the AR device 1028. Alternatively, an MR device could be used in place of the AR device 1028 and a similar user experience can take place, but the user would not be directly viewing physical objects in the environment, such as table 1029, and would instead be presented with a virtual reconstruction of the table 1029 produced from one or more sensors of the MR device (e.g., an outward facing camera capable of recording the surrounding environment).

The user 1002 can use any of the wrist-wearable device 1026, the AR device 1028 (e.g., through physical inputs at the AR device and/or built-in motion tracking of a user's extremities), a smart-textile garment, externally mounted extremity tracking device, the HIPD 1042 to provide user inputs, etc. For example, the user 1002 can perform one or more hand gestures that are detected by the wrist-wearable device 1026 (e.g., using one or more EMG sensors and/or IMUs built into the wrist-wearable device) and/or AR device 1028 (e.g., using one or more image sensors or cameras) to provide a user input. Alternatively, or additionally, the user 1002 can provide a user input via one or more touch surfaces of the wrist-wearable device 1026, the AR device 1028, and/or the HIPD 1042, and/or voice commands captured by a microphone of the wrist-wearable device 1026, the AR device 1028, and/or the HIPD 1042. The wrist-wearable device 1026, the AR device 1028, and/or the HIPD 1042 include an artificially intelligent digital assistant to help the user in providing a user input (e.g., completing a sequence of operations, suggesting different operations or commands, providing reminders, confirming a command). For example, the digital assistant can be invoked through an input occurring at the AR device 1028 (e.g., via an input at a temple arm of the AR device 1028). In some embodiments, the user 1002 can provide a user input via one or more facial gestures and/or facial expressions. For example, cameras of the wrist-wearable device 1026, the AR device 1028, and/or the HIPD 1042 can track the user 1002's eyes for navigating a user interface.

The wrist-wearable device 1026, the AR device 1028, and/or the HIPD 1042 can operate alone or in conjunction to allow the user 1002 to interact with the AR environment. In some embodiments, the HIPD 1042 is configured to operate as a central hub or control center for the wrist-wearable device 1026, the AR device 1028, and/or another communicatively coupled device. For example, the user 1002 can provide an input to interact with the AR environment at any of the wrist-wearable device 1026, the AR device 1028, and/or the HIPD 1042, and the HIPD 1042 can identify one or more back-end and front-end tasks to cause the performance of the requested interaction and distribute instructions to cause the performance of the one or more back-end and front-end tasks at the wrist-wearable device 1026, the AR device 1028, and/or the HIPD 1042. In some embodiments, a back-end task is a background-processing task that is not perceptible by the user (e.g., rendering content, decompression, compression, application-specific operations), and a front-end task is a user-facing task that is perceptible to the user (e.g., presenting information to the user, providing feedback to the user). The HIPD 1042 can perform the back-end tasks and provide the wrist-wearable device 1026 and/or the AR device 1028 operational data corresponding to the performed back-end tasks such that the wrist-wearable device 1026 and/or the AR device 1028 can perform the front-end tasks. In this way, the HIPD 1042, which has more computational resources and greater thermal headroom than the wrist-wearable device 1026 and/or the AR device 1028, performs computationally intensive tasks and reduces the computer resource utilization and/or power usage of the wrist-wearable device 1026 and/or the AR device 1028.

In the example shown by the first AR system 1000a, the HIPD 1042 identifies one or more back-end tasks and front-end tasks associated with a user request to initiate an AR video call with one or more other users (represented by the avatar 1004 and the digital representation of the contact 1006) and distributes instructions to cause the performance of the one or more back-end tasks and front-end tasks. In particular, the HIPD 1042 performs back-end tasks for processing and/or rendering image data (and other data) associated with the AR video call and provides operational data associated with the performed back-end tasks to the AR device 1028 such that the AR device 1028 performs front-end tasks for presenting the AR video call (e.g., presenting the avatar 1004 and the digital representation of the contact 1006).

In some embodiments, the HIPD 1042 can operate as a focal or anchor point for causing the presentation of information. This allows the user 1002 to be generally aware of where information is presented. For example, as shown in the first AR system 1000a, the avatar 1004 and the digital representation of the contact 1006 are presented above the HIPD 1042. In particular, the HIPD 1042 and the AR device 1028 operate in conjunction to determine a location for presenting the avatar 1004 and the digital representation of the contact 1006. In some embodiments, information can be presented within a predetermined distance from the HIPD 1042 (e.g., within five meters). For example, as shown in the first AR system 1000a, virtual object 1008 is presented on the desk some distance from the HIPD 1042. Similar to the above example, the HIPD 1042 and the AR device 1028 can operate in conjunction to determine a location for presenting the virtual object 1008. Alternatively, in some embodiments, presentation of information is not bound by the HIPD 1042. More specifically, the avatar 1004, the digital representation of the contact 1006, and the virtual object 1008 do not have to be presented within a predetermined distance of the HIPD 1042. While an AR device 1028 is described working with an HIPD, an MR headset can be interacted with in the same way as the AR device 1028.

User inputs provided at the wrist-wearable device 1026, the AR device 1028, and/or the HIPD 1042 are coordinated such that the user can use any device to initiate, continue, and/or complete an operation. For example, the user 1002 can provide a user input to the AR device 1028 to cause the AR device 1028 to present the virtual object 1008 and, while the virtual object 1008 is presented by the AR device 1028, the user 1002 can provide one or more hand gestures via the wrist-wearable device 1026 to interact and/or manipulate the virtual object 1008. While an AR device 1028 is described working with a wrist-wearable device 1026, an MR headset can be interacted with in the same way as the AR device 1028.

### Integration of Artificial Intelligence With XR Systems

FIG. 10A illustrates an interaction in which an artificially intelligent virtual assistant can assist in requests made by a user 1002. The AI virtual assistant can be used to complete open-ended requests made through natural language inputs by a user 1002. For example, in FIG. 10A the user 1002 makes an audible request 1044 to summarize the conversation and then share the summarized conversation with others in the meeting. In addition, the AI virtual assistant is configured to use sensors of the XR system (e.g., cameras of an XR headset, microphones, and various other sensors of any of the devices in the system) to provide contextual prompts to the user for initiating tasks.

FIG. 10A also illustrates an example neural network 1052 used in Artificial Intelligence applications. Uses of Artificial Intelligence (AI) are varied and encompass many different aspects of the devices and systems described herein. AI capabilities cover a diverse range of applications and deepen interactions between the user 1002 and user devices (e.g., the AR device 1028, an MR device 1032, the HIPD 1042, the wrist-wearable device 1026). The AI discussed herein can be derived using many different training techniques. While the primary AI model example discussed herein is a neural network, other AI models can be used. Non-limiting examples of AI models include artificial neural networks (ANNs), deep neural networks (DNNs), convolution neural networks (CNNs), recurrent neural networks (RNNs), large language models (LLMs), long short-term memory networks, transformer models, decision trees, random forests, support vector machines, k-nearest neighbors, genetic algorithms, Markov models, Bayesian networks, fuzzy logic systems, and deep reinforcement learnings, etc. The AI models can be implemented at one or more of the user devices, and/or any other devices described herein. For devices and systems herein that employ multiple AI models, different models can be used depending on the task. For example, for a natural-language artificially intelligent virtual assistant, an LLM can be used and for the object detection of a physical environment, a DNN can be used instead.

In another example, an AI virtual assistant can include many different AI models and based on the user's request, multiple AI models may be employed (concurrently, sequentially or a combination thereof). For example, an LLM-based AI model can provide instructions for helping a user follow a recipe and the instructions can be based in part on another AI model that is derived from an ANN, a DNN, an RNN, etc. that is capable of discerning what part of the recipe the user is on (e.g., object and scene detection).

As AI training models evolve, the operations and experiences described herein could potentially be performed with different models other than those listed above, and a person skilled in the art would understand that the list above is non-limiting.

A user 1002 can interact with an AI model through natural language inputs captured by a voice sensor, text inputs, or any other input modality that accepts natural language and/or a corresponding voice sensor module. In another instance, input is provided by tracking the eye gaze of a user 1002 via a gaze tracker module. Additionally, the AI model can also receive inputs beyond those supplied by a user 1002. For example, the AI can generate its response further based on environmental inputs (e.g., temperature data, image data, video data, ambient light data, audio data, GPS location data, inertial measurement (i.e., user motion) data, pattern recognition data, magnetometer data, depth data, pressure data, force data, neuromuscular data, heart rate data, temperature data, sleep data) captured in response to a user request by various types of sensors and/or their corresponding sensor modules. The sensors'data can be retrieved entirely from a single device (e.g., AR device 1028) or from multiple devices that are in communication with each other (e.g., a system that includes at least two of an AR device 1028, an MR device 1032, the HIPD 1042, the wrist-wearable device 1026, etc.). The AI model can also access additional information (e.g., one or more servers 1030, the computers 1040, the mobile devices 1050, and/or other electronic devices) via a network 1025.

A non-limiting list of AI-enhanced functions includes but is not limited to image recognition, speech recognition (e.g., automatic speech recognition), text recognition (e.g., scene text recognition), pattern recognition, natural language processing and understanding, classification, regression, clustering, anomaly detection, sequence generation, content generation, and optimization. In some embodiments, AI-enhanced functions are fully or partially executed on cloud-computing platforms communicatively coupled to the user devices (e.g., the AR device 1028, an MR device 1032, the HIPD 1042, the wrist-wearable device 1026) via the one or more networks. The cloud-computing platforms provide scalable computing resources, distributed computing, managed AI services, interference acceleration, pre-trained models, APIs and/or other resources to support comprehensive computations required by the AI-enhanced function.

Example outputs stemming from the use of an AI model can include natural language responses, mathematical calculations, charts displaying information, audio, images, videos, texts, summaries of meetings, predictive operations based on environmental factors, classifications, pattern recognitions, recommendations, assessments, or other operations. In some embodiments, the generated outputs are stored on local memories of the user devices (e.g., the AR device 1028, an MR device 1032, the HIPD 1042, the wrist-wearable device 1026), storage options of the external devices (servers, computers, mobile devices, etc.), and/or storage options of the cloud-computing platforms.

The AI-based outputs can be presented across different modalities (e.g., audio-based, visual-based, haptic-based, and any combination thereof) and across different devices of the XR system described herein. Some visual-based outputs can include the displaying of information on XR augments of an XR headset, user interfaces displayed at a wrist-wearable device, laptop device, mobile device, etc. On devices with or without displays (e.g., HIPD 1042), haptic feedback can provide information to the user 1002. An AI model can also use the inputs described above to determine the appropriate modality and device(s) to present content to the user (e.g., a user walking on a busy road can be presented with an audio output instead of a visual output to avoid distracting the user 1002).

### Example Augmented Reality Interaction

FIG. 10B shows the user 1002 wearing the wrist-wearable device 1026 and the AR device 1028 and holding the HIPD 1042. In the second AR system 1000b, the wrist-wearable device 1026, the AR device 1028, and/or the HIPD 1042 are used to receive and/or provide one or more messages to a contact of the user 1002. In particular, the wrist-wearable device 1026, the AR device 1028, and/or the HIPD 1042 detect and coordinate one or more user inputs to initiate a messaging application and prepare a response to a received message via the messaging application.

In some embodiments, the user 1002 initiates, via a user input, an application on the wrist-wearable device 1026, the AR device 1028, and/or the HIPD 1042 that causes the application to initiate on at least one device. For example, in the second AR system 1000b the user 1002 performs a hand gesture associated with a command for initiating a messaging application (represented by messaging user interface 1012); the wrist-wearable device 1026 detects the hand gesture; and, based on a determination that the user 1002 is wearing the AR device 1028, causes the AR device 1028 to present a messaging user interface 1012 of the messaging application. The AR device 1028 can present the messaging user interface 1012 to the user 1002 via its display (e.g., as shown by user 1002's field of view 1010). In some embodiments, the application is initiated and can be run on the device (e.g., the wrist-wearable device 1026, the AR device 1028, and/or the HIPD 1042) that detects the user input to initiate the application, and the device provides another device operational data to cause the presentation of the messaging application. For example, the wrist-wearable device 1026 can detect the user input to initiate a messaging application, initiate and run the messaging application, and provide operational data to the AR device 1028 and/or the HIPD 1042 to cause presentation of the messaging application. Alternatively, the application can be initiated and run at a device other than the device that detected the user input. For example, the wrist-wearable device 1026 can detect the hand gesture associated with initiating the messaging application and cause the HIPD 1042 to run the messaging application and coordinate the presentation of the messaging application.

Further, the user 1002 can provide a user input provided at the wrist-wearable device 1026, the AR device 1028, and/or the HIPD 1042 to continue and/or complete an operation initiated at another device. For example, after initiating the messaging application via the wrist-wearable device 1026 and while the AR device 1028 presents the messaging user interface 1012, the user 1002 can provide an input at the HIPD 1042 to prepare a response (e.g., shown by the swipe gesture performed on the HIPD 1042). The user 1002's gestures performed on the HIPD 1042 can be provided and/or displayed on another device. For example, the user 1002's swipe gestures performed on the HIPD 1042 are displayed on a virtual keyboard of the messaging user interface 1012 displayed by the AR device 1028.

In some embodiments, the wrist-wearable device 1026, the AR device 1028, the HIPD 1042, and/or other communicatively coupled devices can present one or more notifications to the user 1002. The notification can be an indication of a new message, an incoming call, an application update, a status update, etc. The user 1002 can select the notification via the wrist-wearable device 1026, the AR device 1028, or the HIPD 1042 and cause presentation of an application or operation associated with the notification on at least one device. For example, the user 1002 can receive a notification that a message was received at the wrist-wearable device 1026, the AR device 1028, the HIPD 1042, and/or other communicatively coupled device and provide a user input at the wrist-wearable device 1026, the AR device 1028, and/or the HIPD 1042 to review the notification, and the device detecting the user input can cause an application associated with the notification to be initiated and/or presented at the wrist-wearable device 1026, the AR device 1028, and/or the HIPD 1042.

While the above example describes coordinated inputs used to interact with a messaging application, the skilled artisan will appreciate upon reading the descriptions that user inputs can be coordinated to interact with any number of applications including, but not limited to, gaming applications, social media applications, camera applications, web-based applications, financial applications, etc. For example, the AR device 1028 can present to the user 1002 game application data and the HIPD 1042 can use a controller to provide inputs to the game. Similarly, the user 1002 can use the wrist-wearable device 1026 to initiate a camera of the AR device 1028, and the user can use the wrist-wearable device 1026, the AR device 1028, and/or the HIPD 1042 to manipulate the image capture (e.g., zoom in or out, apply filters) and capture image data.

While an AR device 1028 is shown being capable of certain functions, it is understood that an AR device can be an AR device with varying functionalities based on costs and market demands. For example, an AR device may include a single output modality such as an audio output modality. In another example, the AR device may include a low-fidelity display as one of the output modalities, where simple information (e.g., text and/or low-fidelity images/video) is capable of being presented to the user. In yet another example, the AR device can be configured with face-facing light emitting diodes (LEDs) configured to provide a user with information, e.g., an LED around the right-side lens can illuminate to notify the wearer to turn right while directions are being provided or an LED on the left-side can illuminate to notify the wearer to turn left while directions are being provided. In another embodiment, the AR device can include an outward-facing projector such that information (e.g., text information, media) may be displayed on the palm of a user's hand or other suitable surface (e.g., a table, whiteboard). In yet another embodiment, information may also be provided by locally dimming portions of a lens to emphasize portions of the environment in which the user's attention should be directed. Some AR devices can present AR augments either monocularly or binocularly (e.g., an AR augment can be presented at only a single display associated with a single lens as opposed presenting an AR augmented at both lenses to produce a binocular image). In some instances an AR device capable of presenting AR augments binocularly can optionally display AR augments monocularly as well (e.g., for power-saving purposes or other presentation considerations). These examples are non-exhaustive and features of one AR device described above can be combined with features of another AR device described above. While features and experiences of an AR device have been described generally in the preceding sections, it is understood that the described functionalities and experiences can be applied in a similar manner to an MR headset, which is described below in the proceeding sections.

### Example Mixed Reality Interaction

Turning to FIGS. 10C-1 and 10C-2, the user 1002 is shown wearing the wrist-wearable device 1026 and an MR device 1032 (e.g., a device capable of providing either an entirely VR experience or an MR experience that displays object(s) from a physical environment at a display of the device) and holding the HIPD 1042. In the third AR system 1000c, the wrist-wearable device 1026, the MR device 1032, and/or the HIPD 1042 are used to interact within an MR environment, such as a VR game or other MR/VR application. While the MR device 1032 presents a representation of a VR game (e.g., first MR game environment 1020) to the user 1002, the wrist-wearable device 1026, the MR device 1032, and/or the HIPD 1042 detect and coordinate one or more user inputs to allow the user 1002 to interact with the VR game.

In some embodiments, the user 1002 can provide a user input via the wrist-wearable device 1026, the MR device 1032, and/or the HIPD 1042 that causes an action in a corresponding MR environment. For example, the user 1002 in the third MR system 1000c (shown in FIG. 10C-1) raises the HIPD 1042 to prepare for a swing in the first MR game environment 1020. The MR device 1032, responsive to the user 1002 raising the HIPD 1042, causes the MR representation of the user 1022 to perform a similar action (e.g., raise a virtual object, such as a virtual sword 1024). In some embodiments, each device uses respective sensor data and/or image data to detect the user input and provide an accurate representation of the user 1002's motion. For example, image sensors (e.g., SLAM cameras or other cameras) of the HIPD 1042 can be used to detect a position of the HIPD 1042 relative to the user 1002's body such that the virtual object can be positioned appropriately within the first MR game environment 1020; sensor data from the wrist-wearable device 1026 can be used to detect a velocity at which the user 1002 raises the HIPD 1042 such that the MR representation of the user 1022 and the virtual sword 1024 are synchronized with the user 1002's movements; and image sensors of the MR device 1032 can be used to represent the user 1002's body, boundary conditions, or real-world objects within the first MR game environment 1020.

In FIG. 10C-2, the user 1002 performs a downward swing while holding the HIPD 1042. The user 1002's downward swing is detected by the wrist-wearable device 1026, the MR device 1032, and/or the HIPD 1042 and a corresponding action is performed in the first MR game environment 1020. In some embodiments, the data captured by each device is used to improve the user's experience within the MR environment. For example, sensor data of the wrist-wearable device 1026 can be used to determine a speed and/or force at which the downward swing is performed and image sensors of the HIPD 1042 and/or the MR device 1032 can be used to determine a location of the swing and how it should be represented in the first MR game environment 1020, which, in turn, can be used as inputs for the MR environment (e.g., game mechanics, which can use detected speed, force, locations, and/or aspects of the user 1002's actions to classify a user's inputs (e.g., user performs a light strike, hard strike, critical strike, glancing strike, miss) or calculate an output (e.g., amount of damage)).

FIG. 10C-2 further illustrates that a portion of the physical environment is reconstructed and displayed at a display of the MR device 1032 while the MR game environment 1020 is being displayed. In this instance, a reconstruction of the physical environment 1046 is displayed in place of a portion of the MR game environment 1020 when object(s) in the physical environment are potentially in the path of the user (e.g., a collision with the user and an object in the physical environment are likely). Thus, this example MR game environment 1020 includes (i) an immersive VR portion 1048 (e.g., an environment that does not have a corollary counterpart in a nearby physical environment) and (ii) a reconstruction of the physical environment 1046 (e.g., table 1050 and cup 1052). While the example shown here is an MR environment that shows a reconstruction of the physical environment to avoid collisions, other uses of reconstructions of the physical environment can be used, such as defining features of the virtual environment based on the surrounding physical environment (e.g., a virtual column can be placed based on an object in the surrounding physical environment (e.g., a tree)).

While the wrist-wearable device 1026, the MR device 1032, and/or the HIPD 1042 are described as detecting user inputs, in some embodiments, user inputs are detected at a single device (with the single device being responsible for distributing signals to the other devices for performing the user input). For example, the HIPD 1042 can operate an application for generating the first MR game environment 1020 and provide the MR device 1032 with corresponding data for causing the presentation of the first MR game environment 1020, as well as detect the user 1002's movements (while holding the HIPD 1042) to cause the performance of corresponding actions within the first MR game environment 1020. Additionally or alternatively, in some embodiments, operational data (e.g., sensor data, image data, application data, device data, and/or other data) of one or more devices is provided to a single device (e.g., the HIPD 1042) to process the operational data and cause respective devices to perform an action associated with processed operational data.

In some embodiments, the user 1002 can wear a wrist-wearable device 1026, wear an MR device 1032, wear smart textile-based garments 1038 (e.g., wearable haptic gloves), and/or hold an HIPD 1042 device. In this embodiment, the wrist-wearable device 1026, the MR device 1032, and/or the smart textile-based garments 1038 are used to interact within an MR environment (e.g., any AR or MR system described above in reference to FIGS. 10A-10B). While the MR device 1032 presents a representation of an MR game (e.g., second MR game environment 1020) to the user 1002, the wrist-wearable device 1026, the MR device 1032, and/or the smart textile-based garments 1038 detect and coordinate one or more user inputs to allow the user 1002 to interact with the MR environment.

In some embodiments, the user 1002 can provide a user input via the wrist-wearable device 1026, an HIPD 1042, the MR device 1032, and/or the smart textile-based garments 1038 that causes an action in a corresponding MR environment. In some embodiments, each device uses respective sensor data and/or image data to detect the user input and provide an accurate representation of the user 1002's motion. While four different input devices are shown (e.g., a wrist-wearable device 1026, an MR device 1032, an HIPD 1042, and a smart textile-based garment 1038) each one of these input devices entirely on its own can provide inputs for fully interacting with the MR environment. For example, the wrist-wearable device can provide sufficient inputs on its own for interacting with the MR environment. In some embodiments, if multiple input devices are used (e.g., a wrist-wearable device and the smart textile-based garment 1038) sensor fusion can be utilized to ensure inputs are correct. While multiple input devices are described, it is understood that other input devices can be used in conjunction or on their own instead, such as but not limited to external motion-tracking cameras, other wearable devices fitted to different parts of a user, apparatuses that allow for a user to experience walking in an MR environment while remaining substantially stationary in the physical environment, etc.

As described above, the data captured by each device is used to improve the user's experience within the MR environment. Although not shown, the smart textile-based garments 1038 can be used in conjunction with an MR device and/or an HIPD 1042.

While some experiences are described as occurring on an AR device and other experiences are described as occurring on an MR device, one skilled in the art would appreciate that experiences can be ported over from an MR device to an AR device, and vice versa.

### Other Interactions

While numerous examples are described in this application related to extended-reality environments, one skilled in the art would appreciate that certain interactions may be possible with other devices. For example, a user may interact with a robot (e.g., a humanoid robot, a task specific robot, or other type of robot) to perform tasks inclusive of, leading to, and/or otherwise related to the tasks described herein. In some embodiments, these tasks can be user specific and learned by the robot based on training data supplied by the user and/or from the user's wearable devices (including head-worn and wrist-worn, among others) in accordance with techniques described herein. As one example, this training data can be received from the numerous devices described in this application (e.g., from sensor data and user-specific interactions with head-wearable devices, wrist-wearable devices, intermediary processing devices, or any combination thereof). Other data sources are also conceived outside of the devices described here. For example, AI models for use in a robot can be trained using a blend of user-specific data and non-user specific-aggregate data. The robots may also be able to perform tasks wholly unrelated to extended reality environments, and can be used for performing quality-of-life tasks (e.g., performing chores, completing repetitive operations, etc.). In certain embodiments or circumstances, the techniques and/or devices described herein can be integrated with and/or otherwise performed by the robot.

Some definitions of devices and components that can be included in some or all of the example devices discussed are defined here for ease of reference. A skilled artisan will appreciate that certain types of the components described may be more suitable for a particular set of devices, and less suitable for a different set of devices. But subsequent reference to the components defined here should be considered to be encompassed by the definitions provided.

In some embodiments example devices and systems, including electronic devices and systems, will be discussed. Such example devices and systems are not intended to be limiting, and one of skill in the art will understand that alternative devices and systems to the example devices and systems described herein may be used to perform the operations and construct the systems and devices that are described herein.

As described herein, an electronic device is a device that uses electrical energy to perform a specific function. It can be any physical object that contains electronic components such as transistors, resistors, capacitors, diodes, and integrated circuits. Examples of electronic devices include smartphones, laptops, digital cameras, televisions, gaming consoles, and music players, as well as the example electronic devices discussed herein. As described herein, an intermediary electronic device is a device that sits between two other electronic devices, and/or a subset of components of one or more electronic devices and facilitates communication, and/or data processing and/or data transfer between the respective electronic devices and/or electronic components.

The foregoing descriptions of FIGS. 10A-10C-2 provided above are intended to augment the description provided in reference to FIGS. 1-9. While terms in the following description may not be identical to terms used in the foregoing description, a person having ordinary skill in the art would understand these terms to have the same meaning.

Any data collection performed by the devices described herein and/or any devices configured to perform or cause the performance of the different embodiments described above in reference to any of the Figures, hereinafter the “devices,” is done with user consent and in a manner that is consistent with all applicable privacy laws. Users are given options to allow the devices to collect data, as well as the option to limit or deny collection of data by the devices. A user is able to opt in or opt out of any data collection at any time. Further, users are given the option to request the removal of any collected data.

It will be understood that, although the terms “first,” “second,” etc. may be used herein to describe various elements, these elements should not be limited by these terms. These terms are only used to distinguish one element from another.

The terminology used herein is for the purpose of describing particular embodiments only and is not intended to be limiting of the claims. As used in the description of the embodiments and the appended claims, the singular forms “a,” “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” can be construed to mean “when” or “upon” or “in response to determining” or “in accordance with a determination” or “in response to detecting,” that a stated condition precedent is true, depending on the context. Similarly, the phrase “if it is determined [that a stated condition precedent is true]” or “if [a stated condition precedent is true]” or “when [a stated condition precedent is true]” can be construed to mean “upon determining” or “in response to determining” or “in accordance with a determination” or “upon detecting” or “in response to detecting” that the stated condition precedent is true, depending on the context.

The foregoing description, for purpose of explanation, has been described with reference to specific embodiments. However, the illustrative discussions above are not intended to be exhaustive or to limit the claims to the precise forms disclosed. Many modifications and variations are possible in view of the above teachings. The embodiments were chosen and described in order to best explain principles of operation and practical applications, to thereby enable others skilled in the art.

What is claimed is:

1. An extended-reality (XR) device comprising:
a frame configured to be worn on a head of a user;
a sensor located on the frame, the sensor configured to collect data corresponding to an attribute of the head of the user; and
a processor programmed to:
receive the data from the sensor;
determine a metric corresponding to an attribute of a body of the user based on the data corresponding to the attribute of the head of the user;
determine whether a difference between the metric and an expected metric corresponding to the attribute of the body of the user satisfies a predetermined threshold; and
responsive to determining that the difference satisfies the predetermined threshold, cause presentation of an indication pertaining to the attribute of the body of the user.

2. The XR device of claim 1, wherein the processor is further programmed to:
receive additional data from the sensor;
determine that the metric has changed based on the additional data corresponding to the attribute of the head of the user;
determine that a second difference between the changed metric and the expected metric pertaining to the attribute of the body of the user does not satisfy the predetermined threshold; and
responsive to determining that the second difference does not satisfy the predetermined threshold, cause presentation of a second indication pertaining to the attribute of the user, wherein the second indication indicates that the second difference does not satisfy the predetermined threshold.

3. The XR device of claim 1, wherein the processor is further programmed to:
receive yet additional data from an additional sensor, distinct from the sensor;
determine an additional metric, distinct from the metric, corresponding to an additional attribute of the body of the user, distinct from the attribute, based on the yet additional data corresponding to the additional attribute and the data corresponding to the attribute of the head of the user;
determine whether an additional difference between the additional metric and an additional expected metric corresponding to the additional attribute of the body of the user satisfies an additional predetermined threshold; and
responsive to determining that the additional difference satisfies the additional predetermined threshold, cause presentation of an indication pertaining to the additional attribute of the user.

4. The XR device of claim 1, wherein:
the sensor comprises an altimeter configured to collect altitude data corresponding to an altitude of the head of the user;
determining the metric corresponding to the attribute of the user comprises determining an altitude metric approximating the altitude of the head of the user based on the altitude data;
the processor is further programmed to:
determine that the user is performing a selected exercise based on the altitude metric; and
determine a motion profile for performance of the selected exercise;
determining whether the difference between the metric and the expected metric satisfies the predetermined threshold comprises determining whether the user is performing the selected exercise in accordance with the motion profile based on the altitude metric; and
the indication pertaining to the attribute of the user indicates whether the user is performing the selected exercise in accordance with the motion profile.

5. The XR device of claim 4, wherein:
the motion profile for the selected exercise requires that the altitude metric reach a particular terminal altitude corresponding to a midpoint in the selected exercise; and
determining whether the user is performing the selected exercise in accordance with the motion profile comprises determining whether a difference between the altitude metric and the particular terminal altitude satisfies an acceptable deviation threshold.

6. The XR device of claim 4, wherein:
the motion profile for the selected exercise requires that the altitude metric oscillates within a particular vertical oscillation threshold; and
determining whether the user is performing the selected exercise in accordance with the motion profile comprises determining whether a vertical oscillation indicated by the altitude metric is maintained within the particular vertical oscillation threshold.

7. The XR device of claim 4, wherein:
the motion profile for the selected exercise requires that the altitude metric have a substantial variation value; and
determining whether the user is performing the selected exercise in accordance with the motion profile comprises determining whether a difference between the altitude metric and a previous altitude metric based on the altitude data is at or above the substantial variation value.

8. The XR device of claim 1, wherein the processor is further programmed to:
determine a performance metric based on a selected exercise and the difference between the metric and the expected metric; and
cause presentation of a real-time indication of the performance metric.

9. The XR device of claim 1, wherein:
the user is performing a selected exercise; and
the processor is further programmed to:
receive physiological data during a period of time from a plurality of sensors included with the XR device, wherein the physiological data comprises one or more of heart rate data, gait data, and pupil dilation data;
receive action data during the period of time from a second plurality of sensors included with the XR device, distinct from the plurality of sensors, wherein the action data comprises one or more of head position data, audio data, and body position data;
determine a plurality of metrics during the period of time, distinct from the metric, corresponding to a plurality of attributes of the body of the user, distinct from the attribute, based on the physiological data and the action data corresponding to the plurality of attributes;
during the period of time, determine whether a plurality of differences between the plurality of metrics and a plurality of expected metrics corresponding to the plurality of attributes of the body of the user satisfies a plurality of additional thresholds; and
responsive to determining that any of the plurality of differences between the plurality of metrics and the plurality of expected metrics corresponding to the plurality of attributes of the body of the user satisfies a plurality of predetermined thresholds at any point in time during the period of time, cause presentation of an indication instructing the user to alter performance of the selected exercise.

10. The XR device of claim 9, wherein:
responsive to determining that none of the plurality of differences between the plurality of metrics and the plurality of expected metrics corresponding to the plurality of attributes of the body of the user satisfies the plurality of predetermined thresholds, cause presentation of an indication encouraging the user to exert more effort in the exercise; and
responsive to determining that any of the plurality of differences between the plurality of metrics and the plurality of expected metrics corresponding to the plurality of attributes of the body of the user satisfies the plurality of predetermined thresholds at any point in time during the period of time, cause presentation of an indication instructing the user to exert less effort in the exercise.

11. The XR device of claim 1, wherein the processor is further programmed to:
determine a further metric corresponding to a physiological attribute of the body of the user based on the data of the attribute of the body of the user;
determine that an emergency event has occurred based on the physiological attribute; and
perform an emergency action based on the emergency event.

12. The XR device of claim 11, wherein the determination that an emergency event has occurred is further based on further data from one or more of a temperature sensor, an inertial measurement unit, an eye-tracking sensor, a wear detector, a camera, and a GPS sensor.

13. The XR device of claim 11, wherein:
the further metric is an acceleration metric approximating an acceleration of the head of the user;
the emergency event comprises the acceleration metric exceeding an emergency acceleration threshold and the user remaining at a position with zero acceleration for a threshold amount of time; and
the emergency action comprises:
causing a camera to capture image data of a field of view of the user; and
sending the image data to one or more of an emergency contact of the user and emergency services.

14. A non-transitory computer-readable storage medium comprising executable instructions that, when executed by one or more processors, cause the one or more processors to:
receive data from a sensor located on a frame, the frame configured to be worn on a head of a user and the sensor configured to collect data corresponding to an attribute of the head of the user;
determine a metric corresponding to an attribute of a body of the user based on the data corresponding to the attribute of the head of the user;
determine whether a difference between the metric and an expected metric corresponding to the attribute of the body of the user satisfies a predetermined threshold; and
responsive to determining that the difference satisfies the predetermined threshold, cause presentation of an indication pertaining to the attribute of the user.

15. The non-transitory computer-readable storage medium of claim 14, wherein the executable instructions, when executed by the one or more processors, further cause the one or more processors to:
receive additional data from the sensor;
determine that the metric has changed based on the additional data corresponding to the attribute of the head of the user;
determine that a second difference between the changed metric and the expected metric pertaining to the attribute of the body of the user does not satisfy the predetermined threshold; and
responsive to determining that the second difference does not satisfy the predetermined threshold, cause presentation of a second indication pertaining to the attribute of the user, wherein the second indication indicates that the second difference does not satisfy the predetermined threshold.

16. The non-transitory computer-readable storage medium of claim 14, wherein the executable instructions, when executed by the one or more processors, further cause the one or more processors to:
receive yet additional data from an additional sensor, distinct from the sensor;
determine an additional metric, distinct from the metric, corresponding to an additional attribute of the body of the user, distinct from the attribute, based on the yet additional data corresponding to the additional attribute and the data corresponding to the attribute of the head of the user;
determine whether an additional difference between the additional metric and an additional expected metric corresponding to the additional attribute of the body of the user satisfies an additional predetermined threshold; and
responsive to determining that the additional difference satisfies the additional predetermined threshold, cause presentation of an indication pertaining to the additional attribute of the user.

17. The non-transitory computer-readable storage medium of claim 14, wherein:
the sensor comprises an altimeter configured to collect altitude data corresponding to an altitude of the head of the user;
determining the metric corresponding to the attribute of the user comprises determining an altitude metric approximating the altitude of the head of the user based on the altitude data;
the executable instructions, when executed by the one or more processors, further cause the one or more processors to:
determine that the user is performing a selected exercise based on the altitude metric; and
determine a motion profile for performance of the selected exercise;
determining whether the difference between the metric and the expected metric satisfies the predetermined threshold comprises determining whether the user is performing the selected exercise in accordance with the motion profile based on the altitude metric; and
the indication pertaining to the attribute of the user indicates whether the user is performing the selected exercise in accordance with the motion profile.

18. A method comprising:
receiving data from a sensor located on a frame, the frame configured to be worn on a head of a user and the sensor configured to collect data corresponding to an attribute of the head of the user;
determining a metric corresponding to an attribute of a body of the user based on the data corresponding to the attribute of the head of the user;
determining whether a difference between the metric and an expected metric corresponding to the attribute of the body of the user satisfies a predetermined threshold; and
responsive to determining that the difference satisfies the predetermined threshold, causing presentation of an indication pertaining to the attribute of the user.

19. The method of claim 18, further comprising:
receiving additional data from the sensor;
determining that the metric has changed based on the additional data corresponding to the attribute of the head of the user;
determining that a second difference between the changed metric and the expected metric corresponding to the attribute of the body of the user does not satisfy the predetermined threshold; and
responsive to determining that the second difference does not satisfy the predetermined threshold, causing presentation of a second indication pertaining to the attribute of the user, wherein the second indication indicates that the second difference does not satisfy the predetermined threshold.

20. The method of claim 18, further comprising:
receiving yet additional data from an additional sensor, distinct from the sensor;
determining an additional metric, distinct from the metric, corresponding to an additional attribute of the body of the user, distinct from the attribute, based on the yet additional data corresponding to the additional attribute and the data corresponding to the attribute of the head of the user;
determining whether an additional difference between the additional metric and an additional expected metric corresponding to the additional attribute of the body of the user satisfies an additional predetermined threshold; and
responsive to determining that the additional difference satisfies the additional predetermined threshold, causing presentation of an indication pertaining to the additional attribute of the user.
